(19) United States
(12) Patent Application Publication
(10) Pub. No.: US 2007/0073278 A1
(43) Pub. Date: Mar. 29, 2007

(54) Cardiac Ablation Dosing

(76) Inventors: Kevin Charles Johnson, Minneapolis, MN (US); Paul Anthony Asselin, Falcon Heights, MN (US); Dana Ray Mester, Oakdale, MN (US)

Correspondence Address: MERCHANT & GOULD PC, P.O. BOX 2903, MINNEAPOLIS, MN, 55402-0903, US

(21) Appl. No.: 11/422,367

(22) Filed: Jun. 6, 2006

Related U.S. Application Data
(63) Continuation-in-part of application No. 11/385,316, filed on Mar. 20, 2006.
(63) Continuation-in-part of application No. 11/385,317, filed on Mar. 20, 2006.
(63) Continuation-in-part of application No. 11/385,358, filed on Mar. 20, 2006.
Application No. 11/385,316 is a continuation-in-part of application No. 11/228,108, filed on Sep. 16, 2005.
Application No. 11/385,317 is a continuation-in-part of application No. 11/228,108, filed on Sep. 16, 2005.
Application No. 11/385,358 is a continuation-in-part of application No. 11/228,108, filed on Sep. 16, 2005.

(51) Int. Cl. A61B 18/18; A61B 18/04

(52) U.S. Cl. 606/11; 606/15; 606/28; 606/42

(57) ABSTRACT

A method and apparatus for applying energy to a target path on a tissue surface includes moving a proximal end of an energy source in a first direction by a first distance. During a first portion of the first distance, the source is in a non-energized mode. During a second portion of the first distance, the source in an energized mode. The proximal end is moved in a second direction along the path opposite the first direction and by a distance less than the first distance. During a first portion of the second direction, the source in a non-energized mode. During a second portion of the second direction, the source in an energized mode. The method compensates for a hysteresis between movement of a distal tip of the energy source and the proximal end.

### I. CROSS-REFERENCE TO RELATED APPLICATIONS

This patent application is a continuation-in-part application of commonly assigned U.S. patent application Ser. Nos. 11/385,316; 11/385,317 and 11/385,358, all filed Mar. 20, 2006 as continuation-in-part applications of commonly assigned U.S. patent application Ser. No. 11/228,108 filed Sep. 16, 2005 titled “Guided Ablation With End-Fire Fiber” (published Apr. 20, 2006 as U.S. patent application Publication No. 2006/0084960).

### II. BACKGROUND OF THE INVENTION

1. Field of the Invention

The present invention relates to surgical instruments for laser cardiac ablation procedures. More particularly, the invention relates to applying ablation energy in a controlled dosage.

2. Description of the Prior Art

A. Atrial Fibrillation

It is known that at least some forms of cardiac arrhythmia are caused by electrical impulses traveling through the cardiac muscle tissue by abnormal routes. In a normal, non-arrhythmic heart, electrical nerve impulses travel in an orderly and well-defined fashion through the sinoatrial node and then through the atrioventricular node in order to create an orderly flow of electrical impulses that lead to contraction in the heart.

In cardiac arrhythmias, cardiac impulses travel along undesirable pathways through the cardiac tissue leading to a rapid heart beat (tachycardia), slow heart beat (bradycardia) or a disorderly heart beat (fibrillation). Atrial fibrillation (AF) is a chaotic heart rhythm of the atrial chambers of the heart. Atrial fibrillation prevents the heart from pumping blood efficiently causing reduced physical activity, stroke, congestive heart failure, cardiomyopathy and death.

B. Maze Procedure—Generally

One technique for treating atrial fibrillation is to surgically create lines in the heart muscle tissue (myocardium) whereby electrical conduction of nerve impulses is blocked or rerouted. This technique for creating lines of electrical blockage is referred to as the Maze procedure.

Initial approaches to performing the Maze procedure involved invasive surgery in which a series of linear incisions are made in the cardiac tissue and then sutured together. The lines of scar tissue that form in the incisions do not conduct electrical impulses and are intended to prevent disorderly contraction of the atrial tissue.

In a typical Maze procedure, up to six non-conductive lines are required. Each of the non-conductive lines is typically several centimeters in length. Once these lines scar and heal, they disrupt electrical pathways that may cause atrial fibrillation. Examples of the Maze procedure and other surgical techniques for treating atrial fibrillation are described in Chiappini, et al., “Cox/Maze III Operation Versus Radiofrequency Ablation for the Surgical Treatment of Atrial Fibrillation: A Comparison Study”, Ann. Thorac. Surg., No. 77, pp. 87-92 (2004) and Cox, “Atrial fibrillation II: Rationale for surgical treatment”, J. Thoracic and Cardiovascular Surg., Vol. 126, No. 6, pp. 1693-1699 (2003).

C. Less Invasive Maze Procedure Technologies

Less invasive ablation techniques have also been utilized to perform the Maze procedure. In such techniques, the surgeon typically drags an a radiofrequency (RF) electrode in a linear fashion along the endocardial (internal) or epicardial (external) surface of the heart to produce a series of lesions using heat to desiccated and ultimately kill cardiac cells. The scaring created by the lesions is ideally contiguous and non-conductive of electrical impulses. For endocardial use, standard ablation catheters or catheters with extended distal electrodes are employed. Epicardially, specially designed handheld probes with a distal electrode for the application of ablating energy are often used.

For the greatest likelihood of success in a Maze procedure, it is particularly important that the lesions created be transmural. A transmural lesion extends through the full wall thickness of the cardiac muscle at the location of the lesion. One factor that limits transmurality of lesions from the epicardium is the cooling effect of blood in and around the heart particularly during ‘off-pump’ procedures during which the heart is beating. This is particularly difficult when radio frequency (RF) energy is employed because it relies exclusively on thermal diffusion to create transmural lesions i.e, flow of heat from higher to lower temperature. The cooling effect of blood on the endocardial surface within the atrium limits attainment of the temperature required to form thermal lesions.

The maximum temperature, at electrode/tissue interface, is also limited to something less than the boiling point of water. Higher temperatures cause boiling of interstitial water creating explosions and subsequent tissue perforations. Perforation of the atrial wall leads to a weakening of the heart structure as well as significant bleeding during surgery that must be controlled.

Additionally, high electrode/tissue temperatures can create burns and adhesion between the probe and the heart tissue. Such adhesions can insulate the probe from the heart tissue blocking the efficient application of energy. These procedures are also a problem for the surgeon and staff who often must stop to clean the tip of the probe.

The efficacy of creating transmural lesions with RF can be enhanced by using a second electrode at the endocardial surface. The endocardial electrode provides a more direct electrical path through cardiac tissue which ‘focuses’ the energy more directly at the target site and secondarily protects the endocardial surface from direct cooling by blood flow in the left atrium. This approach requires access into the left atrium which adds complexity and increases risk to the patient.

The same analysis can also be applied to cryogenic methods which freeze interstitial water causing cellular death. However in this application, the blood warms the tissue at the endocardial surface which again limits the attainment of temperatures required to cause cellular death and create transmural lesions.

A discussion of techniques and technologies for treating atrial fibrillation is set forth in Viola, et al., “The Technology in Use for the Surgical Ablation of Atrial Fibrillation”, Seminars in Thoracic and Cardiovascular Surgery, Vol. 14, No. 3, pp. 198-205 (2002). Viola et al. describe numerous ablation technologies for treating atrial fibrillation with the Maze procedure. These include cryosurgery, microwave energy, radiofrequency energy, and laser ablation.

D. Laser Ablation and the Maze Procedure

The use of lasers in treating atrial fibrillation is desirable because laser energy is first and foremost light which is subsequently converted to heat. Thus, the principles for transmission of light can be used to ‘diffuse’ laser energy in cardiac tissue. At selected wavelengths, light diffusion can be significantly faster and penetrate more deeply than thermal diffusion. To achieve this effect, it is important to understand the spectral characteristics of atrial tissue and select a laser wavelength with high transmissivity, i.e., low absorption. Wavelengths in the near infrared region, 700-1200 nanometers are suitable for achieving such results. Ideally the wavelength would be 790 to 830 or 1020 to 1140 nanometers. As a result, laser ablation is fast and results in narrow lesions. Viola, et al., “The Technology in Use for the Surgical Ablation of Atrial Fibrillation”, Seminars in Thoracic and Cardiovascular Surgery, Vol. 14, No. 3, pp. 201, 204 (2002). However, in the prior art, laser ablation for treating atrial fibrillation has been troublesome.

Viola et al. discuss problems associated with the use of laser energy to treat atrial fibrillation. These concerns are directed to safety and reliability and note that lasers are prone to overheating because of the absence of a self-limiting mechanism. The authors note that over-heating with lasers can lead to crater formation and eventually to perforation, especially when using pin-tip devices. Viola, et al., supra, at p. 203. The authors note that the high power of laser ablation (described as 30 to 80 Watts) results in the laser technique not being widely clinically applied. Id., at p. 201. The mechanical effects resulting from direct heating of the myocardial tissue with laser energy results in cellular explosions caused by shock waves. Viola, et al., supra, at p. 201.

The possibility for perforation of the myocardium with laser energy raises a particular concern for treating atrial fibrillation. The myocardial wall of the atria is quite thin (e.g., about 2 mm in thickness in some locations). A coring of the myocardium by a laser could result in a full wall thickness perforation and resulting leakage of blood.

Viola et al. note the development of a long probe laser that allows diffusion of the laser thermal energy over the long probe tip in a unidirectional fashion. Id., at p. 201. While not mentioning the source of this long probe tip, it is believed by the present inventors to be referring to the atrial fibrillation laser of CardioFocus, Inc., Norton, Mass. (USA) as described in U.S. Patent Application Publication No. 2004/6333A1 in the name of Arnold, et al. (published Jan. 8, 2004) and U.S. Pat. No. 6,579,285 issued to Sinosky. This technology as practiced differs in two ways to that disclosed in the preferred embodiment of the present invention. First, and most importantly, it defocuses the coherent laser beam by using reflective particles to scatter the light longitudinally and radially before it enters the tissue. This reduces the longitudinal movement required to produce linear lesions but, by decreasing the coherency of the laser beam before entering cardiac tissue, and negates many of the advantages of light to more deeply penetrate cardiac tissue. Secondly, this technology uses laser light in the 910 to 980 nanometer wavelengths which has a significant water absorption peak compared to 810 and 1064. The higher absorption reduces the penetration of the laser light through cardiac tissue. Reducing energy penetration depths increases the risk (particularly on a beating heart) of creating a lesion that is less than transmural.

E. Conductivity Verification

A further difficulty with creating linear nonconductive lesions is the inability to verify that a truly nonconductive lesion has been produced. If a transmural lesion is not properly formed in accordance with the Maze procedure, the treatment for atrial fibrillation may not be successful. This could require a second surgical procedure. If the surgeon can promptly discern whether a particular linear lesion is truly non-conducting at the time of the original procedure, correction could be made at the time of treatment. A method of assessing lesion transmurality is described in U.S. patent application Publication No. US 2005/0209589 A1 published Sep. 22, 2005.

F. Placing and Guiding an Atrial Ablation Tool

U.S. patent application Publication No. US2005/0096643 A1 published May 5, 2005 describes formation of a lesion pattern by a surgeon moving the tip of a wand over the heart surface. Use of a tool to guide or control an ablation tool has been suggested. For example, U.S. Pat. No. 6,579,285 (assigned to CardioFocus, Inc.) shows a diffused light fiber tip in a malleable housing. The housing is bent to form a desired shape and placed against the heart. The diffused light fiber tip is moved through the housing in a series of steps to form a lesion. The lesion is formed by stopping the fiber at a location, energizing the motionless fiber to create a lesion, and moving the fiber to a new location to form a subsequent lesion segment. A similar arrangement for an ablation tool is shown in U.S. patent publication No. 2002/0087151 published Jul. 4, 2002 (assigned to AFx, Inc.).

U.S. patent publication No. 2004/0102771 published May 27, 2004 (assigned to Estech, Inc.) describes a device to guide an ablation tool while maintaining contact between the heart and an ablation device. Other devices for either guiding an ablation element or for maintaining contact for between an ablation element and the heart are shown in U.S. Pat. No. 6,237,605 (assigned to Epicor, Inc.). U.S. Pat. No. 6,237,605 describes using vacuum against an epicardium or an inflatable balloon against a pericardium to maintain ablation devices in a fixed position against the heart. U.S. Pat. Nos. 6,514,250 and 6,558,382 (both assigned to Medtronic, Inc.) describe suction to hold ablation elements against a heart.

Commonly assigned U.S. patent application Publication No. US 2005/0182392 A1 published Aug. 18, 2005, teaches a guided ablation apparatus with a laser emitting ablation element mounted in a carriage advanced through a flexible guide member mounted on the heart.

When moving an ablation element in a guided ablation apparatus, a physician cannot visually inspect the location and rate of movement of an ablation element relative to heart tissue. It is an object of the present to provide a guided ablation with enhanced control.

G. Dose Control

When applying ablation energy to heart tissue, the amount of energy applied the tissue (i.e., the energy dose), is important to control. If too much energy is applied, the tissue can carbonize or perforate. If too little energy is applied, a resulting lesion will not be transmural. The present invention is directed to a method and apparatus to control such dosage.

### III. SUMMARY OF THE INVENTION

According to a preferred embodiment of the present invention, a method and apparatus are disclosed for applying energy to a target path on a tissue surface. For example, the target tissue is atrial tissue of a patient's heart and ablative energy is applied to the tissue to create an in situ lesion without perforation of the atrium. The method uses a movable source of energy having a distal end for applying energy to the tissue opposing the distal end. The energy source has a proximal end with an initiation of movement of the distal end delayed from an initiation of movement of the proximal end by a variable amount of discrepancy which is less than a predetermined maximum discrepancy. The distal end is guided for movement along the target path in response to movement of the proximal end.

For example, the source of energy is an optical fiber coupled to a laser energy source. A fiber tip is contained within a guide member. The tip is moved within the guide member by pushing or pulling on a proximal end of the fiber. When a push or pull is initiated, there is a delay (a hysteresis) resulting in no movement of the distal tip. The method of the invention includes moving the proximal end in a first direction by a first distance. During a first portion of the first distance, the source is in a non-energized mode. During a second portion of the first distance, the source in an energized mode. The source is moved in a second direction along the path opposite the first direction and by a distance less than the first distance. During a first portion of the second direction, the source in a non-energized mode. During a second portion of the second direction, the source in an energized mode.

### IV. BRIEF DESCRIPTION OF THE DRAWINGS

FIGS. 1-17 are FIGS. 1-17 of U.S. patent application Ser. Nos. 11/385,316; 11/385,317 and 11/385,358 (the “parent applications”);

FIG. 1 is a side elevation view of a distal portion of a guided ablation apparatus according to the present invention and with a sidewall partially removed to expose interior components;

FIG. is a view of the distal portion of FIG. 1 curved into a loop with an intermediate connector holding the shape of the apparatus;

FIG. 1B is a view taken along lines 1B-1B in FIG. 1A;

FIG. 2 is a bottom plan view of a portion of the apparatus of FIG. 1;

FIG. 3 is a cross sectional view of a guide member of the apparatus of FIG. 1 and showing a carriage in the guide member;

FIG. 4 is a view of FIG. 3 (without carriage) with an optional fabric cover;

FIG. 5 is a longitudinal, cross-sectional, schematic view of the distal portion of FIG. 1;

FIG. 6 is a longitudinal, cross-sectional, schematic view of a proximal portion of a guided ablation apparatus;

FIG. 7 is a top plan view of the apparatus of FIG. 1 shown in a straight alignment;

FIG. 8 is the view of FIG. 7 with the apparatus shown curved;

FIG. 9 is the view of FIG. 7 with the apparatus shown twisted;

FIG. 10 is a schematic representation of internal components of the apparatus of FIG. 1 in a first positioning;

FIG. 11 is the view of FIG. 10 with the components shown in a second positioning;

FIG. 12 is the view of FIG. 10 with the apparatus shown in a third positioning;

FIG. 13 is a side sectional view of the carriage for use in the apparatus;

FIG. 13A is a cross sectional view of a channel in a region of a curved portion of a fiber shown in FIG. 13;

FIG. 13B is the view of FIG. 13A taken in a region of a straight segment of the fiber shown in FIG. 13;

FIG. 13C is the view of FIGS. 13A and 13B taken in a length of the fiber of FIG. 13 with a jacket removed to expose a fiber core;

FIG. 14 is a perspective view of the carriage;

FIG. 15 is a perspective longitudinal sectional view of the carriage;

FIG. 16 is a perspective view (partially schematic) of a control apparatus;

FIG. 17 is FIG. 24A from U.S. patent application Ser. No. 11/228,108 (the “'108 application”) which is a schematic representation of divergence of a laser beam from an end of a fiber;

FIG. 18 is FIG. 25 from the '108 application which is a perspective view of an apparatus according including a guide member and a separate carriage (with side panel removed) for advancing a fiber through the guide member;

FIG. 19 is FIG. 26 from the '108 application which is a front, top and right side perspective view of the carriage of FIG. 18;

FIG. 20 is FIG. 27 from the '108 application which is a top plan view of the carriage of FIG. 19;

FIG. 21 is FIG. 28 from the '108 application which is a side elevation view of the carriage of FIG. 19 with a side panel removed;

FIG. 22 is FIG. 29 from the '108 application which is a front elevation view of the carriage of FIG. 19;

FIG. 23 is FIG. 29A from the '108 application which is a view taken along line 23-23 of FIG. 22;

FIG. 24 is FIG. 30 from the '108 application which is a perspective view of a guide member and carriage according to an alternative embodiment of the invention of the '108 application;

FIG. 25 is FIG. 31 from the '108 application which is a top plan view of the guide member and carriage of FIG. 24;

FIG. 26 is FIG. 32 from the '108 application which is a side elevation view of a guide member and carriage of FIG. 24;

FIG. 27 is FIG. 33 from the '108 application which is a front view showing a carriage received within a guide member of FIG. 24;

FIG. 28 is FIG. 33A from the '108 application which is a view taken along line 28-28 of FIG. 27;

FIG. 29 is FIG. 34 from the '108 application which is a perspective view of a still further embodiment of a guide member and fiber according to the invention of the '108 application;

FIG. 30 is FIG. 35 from the '108 application which is an end view of the apparatus of FIG. 29;

FIG. 31 is FIG. 36 from the '108 application which is a view taken along line 31-31 of FIG. 30;

FIG. 32 is FIG. 37 from the '108 application which is a perspective view of a yet further embodiment of a guide member and fiber according to the invention of the '108 application;

FIG. 33 is FIG. 38 is an end view of the apparatus of FIG. 32;

FIG. 34 is FIG. 39 from the '108 application which is a view taken along line 34-34 of FIG. 33;

FIG. 35 is a graphical representation of multiple passes of a fiber tip over tissue in two zones without accounting for hysteresis;

FIG. 36 is a graphical representation of cumulative dosage within zones following the treatment of FIG. 35;

FIG. 37 is a graphical representation of multiple passes of a fiber tip over tissue in two zones with an accounting for hysteresis and assuming actual hysteresis equals an assumed hysteresis;

FIG. 38 is a graphical representation of cumulative dosage within zones following the treatment of FIG. 37;

FIG. 39 is a graphical representation of multiple passes of a fiber tip over tissue in two zones with an accounting for hysteresis and assuming actual hysteresis equals zero;

FIG. 40 is a graphical representation of cumulative dosage within zones following the treatment of FIG. 39;

FIG. 41 is a graphical representation of fiber movement and power states according to a preferred embodiment of the present invention and assuming an actual hysteresis equals an assumed maximum hysteresis;

FIG. 42 is a graphical representation of fiber movement and power states according to a preferred embodiment of the present invention and assuming an actual hysteresis equals zero;

FIG. 43 is a graphical representation of multiple passes of a fiber tip over tissue in according to the present invention and having a fiber movement and power state according to FIG. 41;

FIG. 44 is a graphical representation of cumulative dosage along a treatment path following the treatment of FIG. 43;

FIG. 45 is a graphical representation of multiple passes of a fiber tip over tissue in according to the present invention and having a fiber movement and power state according to FIG. 42;

FIG. 46 is a graphical representation of cumulative dosage along a treatment path following the treatment of FIG. 45; and

FIG. 46 is a schematic representation of a guide member layout for achieving uniform dosage in a target treatment zone.

### V. DETAILED DESCRIPTION OF THE PREFERRED EMBODIMENTS

Referring now to the several drawing Figures in which identical elements are numbered identically throughout, a description of a preferred embodiment of the present invention will now be provided. In the preferred embodiment, the invention is described with reference to use of a lesion formation tool for applying laser energy to the epicardial surface of the heart to create a transmural ablation line along the heart. As used in this application, the term “ablation” is used in the context of creating necrosed tissue in the myocardium while avoiding tissue perforation or removal. In the following description, a guide member is described for guiding a lesion formation tool in a Maze pattern. It will be appreciated the teachings of the present application could be applied to other types of ablation tools (e.g., RF ablation, ultrasound or other). Also, this application may refer to a lesion as “linear”. The use of “linear” is not meant to be limited to a straight line but is intended to include a curved or other lesion pattern which is elongated and narrow in width.

Unless otherwise described in reference to a preferred embodiment, all components of the invention can be formed of any suitable material subject to ability of such material to withstand the rigors of sterilization and meet all biocompatibility and other requirements of applicable medical device regulations.

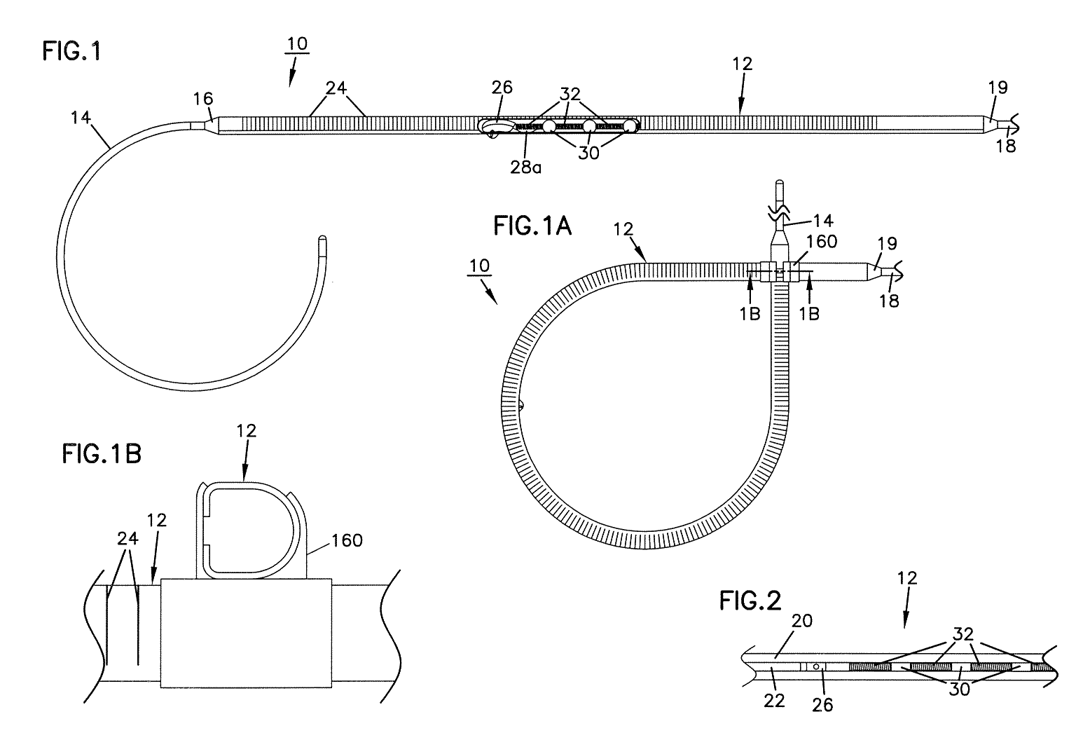

As will be discussed below in Section C “Additional Disclosure of the Present Application”, the present invention is directed to controlling a dose of ablation energy applied to tissue. In a preferred embodiment, the present invention is utilized in a guided laser ablation process as described in the parent applications from which this application claims priority in part. More particularly, the present invention is most preferably used in a guided laser ablation system described with reference to FIGS. 1-17 and described in Section B(b) “Disclosure of Ser. Nos. 11/385,316; 11/385,317 and 11/385,358”. While such is a preferred embodiment, it will be appreciated the present invention is applicable to other ablation energy sources including radio-frequency ablation, thermal ablation, ultrasound ablation and cryogenic ablation.

A. Teachings of Prior Publications

a. Laser Ablation

The aforementioned U.S. patent application Publication. No. US2005/0096643 A1 published May 5, 2005 (incorporated herein by reference) describes, in detail, a surgical wand for applying laser energy to either the epicardial or endocardial surface of the heart. For treating atrial fibrillation through the Maze procedure, the wand preferably emits laser energy as coherent light in a wavelength selected to have a very low absorption and very high scatter in myocardial tissue.

Any wavelength suitable to create necrosed tissue in the myocardium without tissue removal could be used. In a preferred embodiment, the wavelength is a near-infrared wavelength selected to have a very low absorption and very high scatter in myocardial tissue. Biological tissue (such as the myocardium) is largely water. Wavelengths in the ranges of between about 470 to about 900 nanometers and between about 1050 to about 1150 nanometers are known to penetrate water with low absorption (e.g., less than about 30% absorption). Lasers in Cardiovascular Medicine and Surgery: Fundamentals and Techniques, George S. Abela, M. D., Editor, Kluwer Academic Publishers, 101 Philip Drive, Assinippi Park, Norwell, Mass. 02061 USA, p. 28 (1990). More preferably, the wavelength is selected from the ranges of 790 to 850 nanometers (which range corresponds to commercially available medical diode lasers) and 1050 to 1090 nanometers (which range corresponds to Nd:YAG lasers commonly used in other medical procedures).

A laser energy source with a wavelength selected from the above ranges will penetrate the full thickness of the myocardium and result in a transmural lesion (i.e., a full-thickness necrosis of myocardial tissue in the atrium). Further, such a wavelength minimizes carbonization of the tissue and perforation of the myocardial tissue. Such laser emissions are substantially coherent.

In the aforesaid U.S. patent application Publication. No. US2005/0096643, the wand is a hand-held device with a distal tip placed against either the epicardial or endocardial surface of the heart. The wand is manipulated so that the distal tip moves along the surface of the heart to create a Maze lesion of a desired pattern.

b. Guided Member

In U.S. patent application Publication. No. US 2005/0182392 A1 (the “'392 publication”) (incorporated herein by reference), a guide apparatus includes a guide member of an elongated flexible body and having a generally flat bottom surface. A guide channel is formed as a groove centrally positioned within the bottom wall and extending along the longitudinal length of the guide member. A guide carriage is slidably received within the guide channel. The carriage carries a laser emitting tip.

The guide carriage may axially slide within the guide channel but is prevented from moving transverse to its sliding axis as well as being prevented from rotating about the axis. The carriage includes a bottom opening or window. The window may be an open area to pass both emitted light and a flushing fluid or may be a closed window of material selected to pass the wavelength of the emitted light.

A flexible fluid conduit is connected to a proximal end of the carriage. The conduit moves with the carriage within the channel. Pushing the conduit moves the carriage distally. Retraction of the conduit moves the carriage proximally.

An optical fiber passes through the conduit. Spacers hold the fiber coaxially within the conduit with opposing surfaces of the fiber and conduit defining an annular lumen into which cooling fluid from pump may be passed. The fluid both cools components as well as flushing debris which might otherwise accumulate between the fiber and the epicardial surface.

The fiber is carried in the carriage with a distal tip of the fiber positioned to discharge light through the window. Cooling fluid from lumen can also pass through the window. To enhance the atraumatic nature of the carriage, the carriage of the '392 application is formed of a material having a low coefficient of friction or lubricious-like nature against the heart tissue.

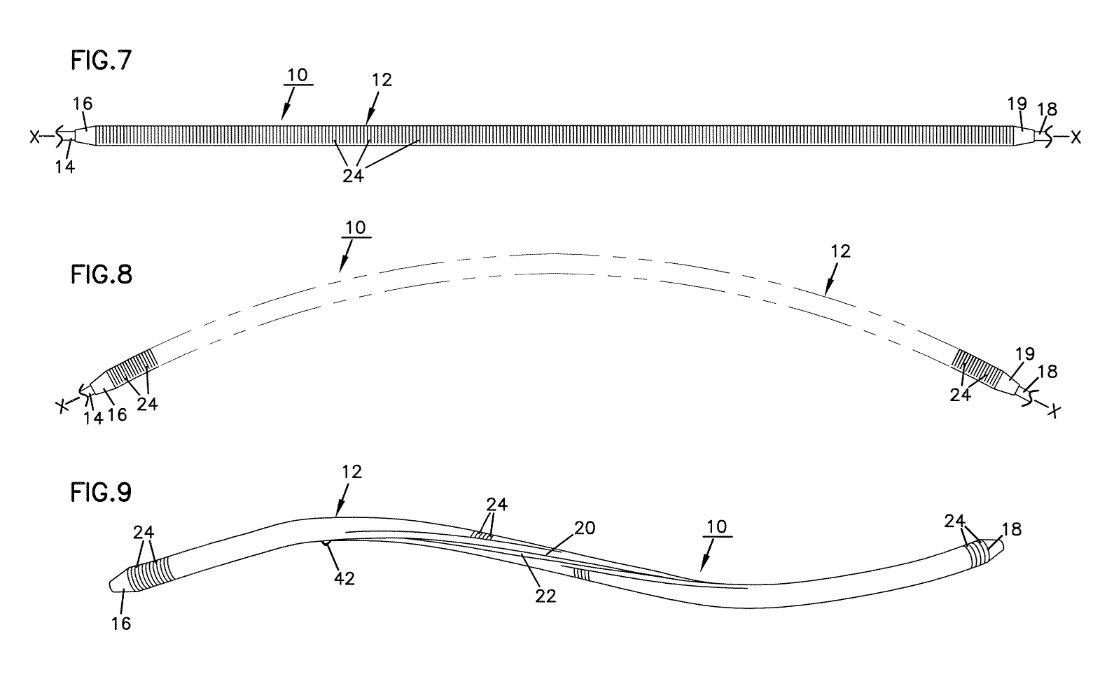

The light from the fiber passes through the window in a light path generally perpendicular to the axis and the plane of the guide member bottom surface. As schematically shown in FIG. 7 of the '392 publication, the end of the fiber is cleaved, polished and coated for the fiber to be a so-called “side fire” laser such that the fiber is not bent. While it is described as preferred that the light from the tip impinge upon the heart tissue at a 90-degree angle, the angle can be varied and still provide therapeutic benefit. Side-fire fibers are well known. A representative example of such is shown in U.S. Pat. No.5,537,499 to Brekke issued Jul. 16, 1996 (incorporated herein by reference).

c. Placement of Guide Member and Formation of Maze Lesions

The guide member is placed on the heart surface and shaped in a desired pattern (e.g., encircling the pulmonary veins on the left atrium). So positioned, the carriage may be moved within the guide channel. The laser fiber may be energized by activating a power source to form a transmural lesion in the heart wall.

The conduit is pushed or pulled as desired to move the carriage distally or proximally, respectively, thereby moving the fiber tip in a desired pattern over the epicardial surface of the heart. The physician moves the carriage along the exterior surface of the heart in order to create lines of ablated (i.e., non-electrically conducting) tissue by raising the temperature of the cardiac tissue to that required to achieve cellular death (typically about 55° C.). It is estimated that, with an operating laser power of about 25 watts, a surgeon can create an ablation line by gliding the moving the carriage over the heart surface at a rate of between about 1 to 5 cm of linear travel per minute. By way of non-limiting example, with a diode laser, power can range from about 5 to about 50 Watts.

While a lesion can be formed by pulling the fiber distally in one pass, it is presently preferred to form the lesion in zones. For example, a desired lesion pattern can be divided into multiple zones. Within a zone, the energized fiber tip is moved back and forth with carriage in the guide member multiple times to apply a desired dosage of energy to tissue in the zone. The carriage and fiber tip are then moved to the next zone and the procedure is repeated.

Throughout this pattern, the carriage holds the laser tip in a constant spacing from the epicardial surface of the heart. The guide member maintains a desired spacing between the end of the ablation tool (i.e., the fiber tip in a preferred embodiment) and the surface of the heart throughout the length of the guide member and avoids direct contact of the ablation member and the heart.

It is desirable to have as close a spacing of the fiber discharge tip to the bottom wall of the guide member as possible to maximize laser energy penetration of myocardial tissue. The power density impinging on cardiac tissue decreases rapidly with increasing spacing. However, a small spacing from the surface of the heart is desirable to prevent coagulation of biological products onto the face of the optical fiber. Build-up of tissue is undesirable. It can cause carbonization and spalling of the optical fiber face which reduces laser energy output from the optical fiber. If sufficient biological material is present in the vicinity of the optical fiber face, overheating and subsequent melting of components can occur. Due to the unobstructed path from the fiber tip to the heart surface, the light is a non-diffused or unmodified beam directed at the heart surface either perpendicularly of at an angle as described above.

The flow of coolant fluid from the window cools the material of the carriage, washes biological material (e.g., blood, tissue debris or the like) from the light path between optical fiber tip and the heart surface, and acts as a lubricant to further facilitate atraumatic gliding movement of the carriage over the surface of the heart.

The washing action of the fluid maximizes the laser energy impinging on the surface of the heart. Additionally, this fluid provides a means to cool the tissue in the region of the carriage to help ensure that tissue carbonization and subsequent vaporization of cardiac tissue do not occur. This substantially reduces the likelihood of perforation of the heart wall. Also, the fluid forms a protective layer at the discharge tip of optical fiber which reduces the likelihood biological residue will impinge on and/or adhere to the discharge tip which can otherwise cause spalling of the fiber tip and reduce optical transmission of laser energy.

Since the fluid flows into the body of the patient, the fluid should be medical grade and biocompatible. Also, the fluid should have a low absorption of the laser energy. A preferred fluid is a physiological saline solution which may be supplied at ambient temperature.

B. Parent Applications

a. Teachings of Parent U.S. patent application Ser. No. 11/228,108

Commonly assigned and co-pending U.S. patent application Ser. No. 11/228,108 (filed Sep. 16, 2005) (the “'108 application”) teaches a guided atrial ablation with an end-fire laser. FIGS. 18-34 are taken from the '108 application and the text of this section is substantially taken from the '108 application to facilitate an understanding of the present invention.

U.S. Patent Application Publication No. U.S. 2005/0182392 A1 (published Aug. 18, 2005) describes a guided laser ablation with a side-fire laser fiber. The light emerges from the fiber distal tip at an angle (60-90 degrees) to the axis of the fiber at the tip.

A side-fire laser fiber permits a low-profile assembly. A guided fiber assembly can be placed in the pericardial space. Since the laser fiber axis is substantially parallel to the tissue surface, the assembly occupies very little space and can readily fit into the pericardial space. In contrast, a wand with an end-fire fiber requires greater surgical access and dissection of the pericardium.

Unfortunately, side-fire laser fibers are subject to performance limitations. Side-fire lasers are subject to possible performance degradation particularly in use with small fibers (such as 400 micron and 0.37 numerical aperture fibers) coupled to an 810-nanometer diode laser. In such applications, the beam ejected from the side-fire laser fiber is not narrow and a substantial portion of the energy is reflected from the surface of the tissue. The teachings of the '108 application combine the benefits of a guided laser application together with a more direct end-fire laser.

The '108 application embodiments that follow describe an end-fire laser fiber in a low-profile apparatus for minimizing the thickness of the apparatus thereby enjoying the optical benefits of end-fire fibers and the size benefits of a side-fire fiber. Further, such embodiments optimize a spacing of a fiber tip from target tissue to enjoy good power density while also enjoying the benefits of flushing and cooling.

An important issue in penetration of laser energy into myocardial tissue is the power density of the incident laser beam. Power density is a ratio of total power exiting an optical fiber to the irradiated surface area or the delivered power per unit area. Higher power densities by definition are more focused and result in narrower and deeper lesions.

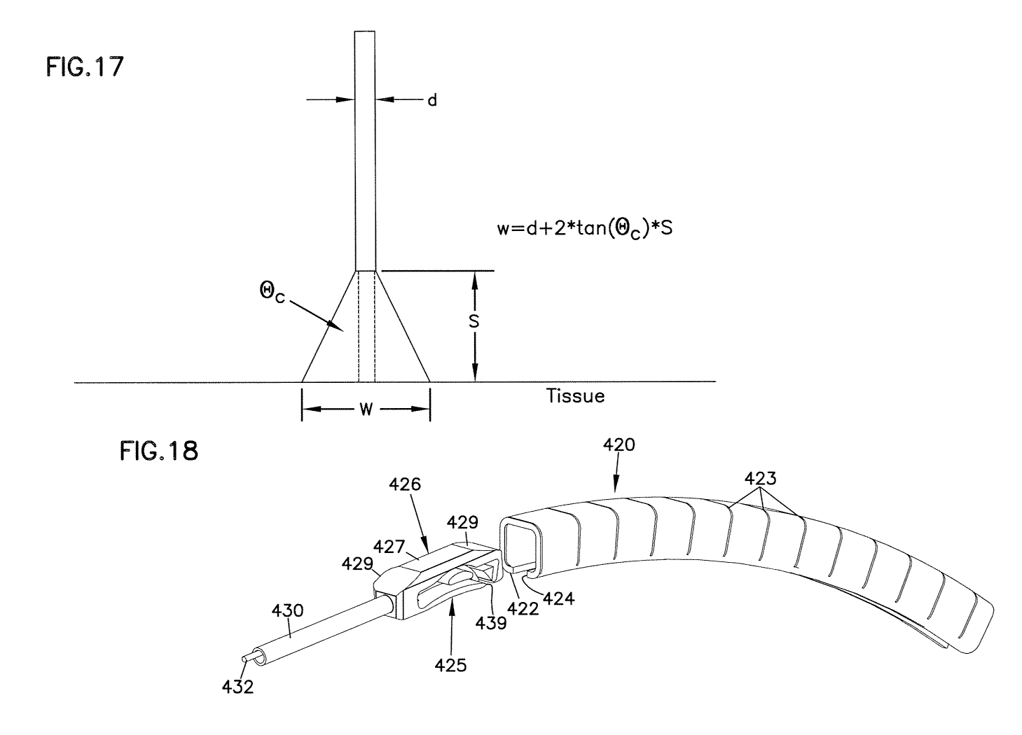

The power density is highest at the exit plane of the optical fiber and decreases as the beam diverges. A beam generally expands in a conical shape with a divergence angle equal to the inverse sine of the numerical aperture of the optical fiber as shown in FIG. 17.

As the spacing between the optical fiber and tissue increases, the power density of the incident laser beam decreases by the square of the distance. Table 1 shows such a decrease for different tissue spacing and optical fiber diameters.

Power density can impact laser tissue interaction in two ways. First, there is a critical power density beyond which the interaction no longer obeys the laws of classical physics and becomes non-linear. This effect disperses laser energy more rapidly into myocardial tissue creating deeper lesions. Second, higher power densities compensate more easily for the parasitic losses associated with absorption of laser energy during passage through myocardial tissue and the cooling effect of blood flow at the endocardial surface increasing the probability of creating transmural lesions on a beating heart. For most atrial applications, power densities above 1000 W/cm² are desirable for creating transmural lesions.

Power density is a more important design consideration for diode lasers because of the higher incidence angle of laser energy at the input end of the optical fiber. The incidence angle is increased as light traverses the optical fiber until reaching the critical angle of the optical fiber. As an example, most commercial diode lasers have incidence angles of 20 degrees requiring the use of optical fibers with a numerical aperture (NA) of 0.37. This yields a divergence angle 22 degrees at the exit plane of the optical fiber resulting in an increased image diameter equal to the distance from the tissue surface (total cone angle is 2× divergence angle). Table 1 shows a 100-fold decrease in power density with only 3/16 inch spacing (about 4.25 mm) from the tissue.

TABLE 1
Effect of Power Density (W/cm²) (0.37 NA Optical Fiber)

| | | |
|---|---|---|
| Tissue Spacing | Tissue Spacing | Fiber Diameter (microns) |

| (mm) | (in) | 400 | 600 |
|---|---|---|---|
| 0.00 | 0.000 | 19,894 | 8,842 |
| 0.25 | 0.010 | 8,872 | 4,986 |
| 0.50 | 0.020 | 4,999 | 3,196 |
| 0.75 | 0.030 | 3,203 | 2,222 |
| 1.00 | 0.039 | 2,226 | 1,634 |
| 1.25 | 0.049 | 1,636 | 1,251 |
| 1.50 | 0.059 | 1,253 | 989 |
| 1.75 | 0.069 | 990 | 802 |
| 2.00 | 0.079 | 802 | 663 |
| 2.25 | 0.089 | 663 | 557 |
| 2.50 | 0.098 | 557 | 475 |
| 2.75 | 0.108 | 475 | 409 |
| 3.00 | 0.118 | 410 | 357 |
| 3.25 | 0.128 | 357 | 313 |
| 3.50 | 0.138 | 314 | 278 |
| 3.75 | 0.148 | 278 | 248 |
| 4.00 | 0.157 | 248 | 222 |
| 4.25 | 0.167 | 222 | 201 |

While direct contact (tissue spacing of 0.00 mm) achieves the greatest power density, it may not be the optimal spacing for ablation of atrial tissue. Higher power densities have a greater potential to carbonize and perforate cardiac tissue especially thinner atrial myocardium. Other variables such as the cooling created by flushing fluids and the divergence angle of the laser light help mitigate the risk of perforations. As will be described, a spacing of about 0.05 inches (about 1.3 mm) achieves a preferred balance of these variables. However, it is believed a spacing between a minimum of 0.25 mm and a maximum of 2.0 mm are acceptable. While greater spacing is possible, the power density drops off significantly and may not be adequate to compensate for the cooling effects on the endocardial surface.

It is possible that effective lesions can be created at a distance greater than 2.0 mm. Myocardial anatomy and positioning of ablation guide along a myocardial surface could result in spacing greater than 2.0 mm from the surface of the tissue, though the flexibility of the guide and follower assembly is such to minimize this distance. Even if 2.0 mm is exceeded, a lesion may still be created at greater than 5 mm spacing, though of less depth. This effect of spacing resulting in decreased penetration and lesion depth may be further minimized by water coupling of optical fiber face to the tissue surface. Water coupling may be enhanced by increased flow rate of flushing solution or by distribution of flow over a greater surface area.

FIG. 18 illustrates a guided laser which avoids use of a side-fire laser. In FIG. 18, the guide member 420 is a biocompatible plastic material formed with a hollow, square-shaped configuration. The guide member 420 has a bottom wall 422 with a slot 424 extending through the bottom wall throughout the length of the guide member 420.

In the embodiment shown in FIG. 18, no apparatus is shown for fixing the guide member 420 against tissue (such as a vacuum attachment as described in the aforemention U.S. patent application Publication No. US 2005/0182392 A1). However, it will be appreciated that the guide member 420 could be provided with such attachment mechanisms as well as with visualization equipment.

Preferably, the guide member 420 includes a feature to permit controlled bending of the guide member. In the embodiment shown, this mechanism is a plurality of slits 423 formed through the guide member along its length. The plane defined by each slit 423 is perpendicular to the longitudinal axis of the guide member 420. The slits 423 are formed through all but the bottom wall 422.

The slits 423 permit the guide member 420 to be bent along the surface of the bottom surface 422 as shown in FIG. 18. As an alternative to slits 423, the guide member 420 can be otherwise reinforced (e.g., through material selection or geometry to accomplish such controlled bending). It is preferred the guide member 420 not be stretchable along its longitudinal axis.

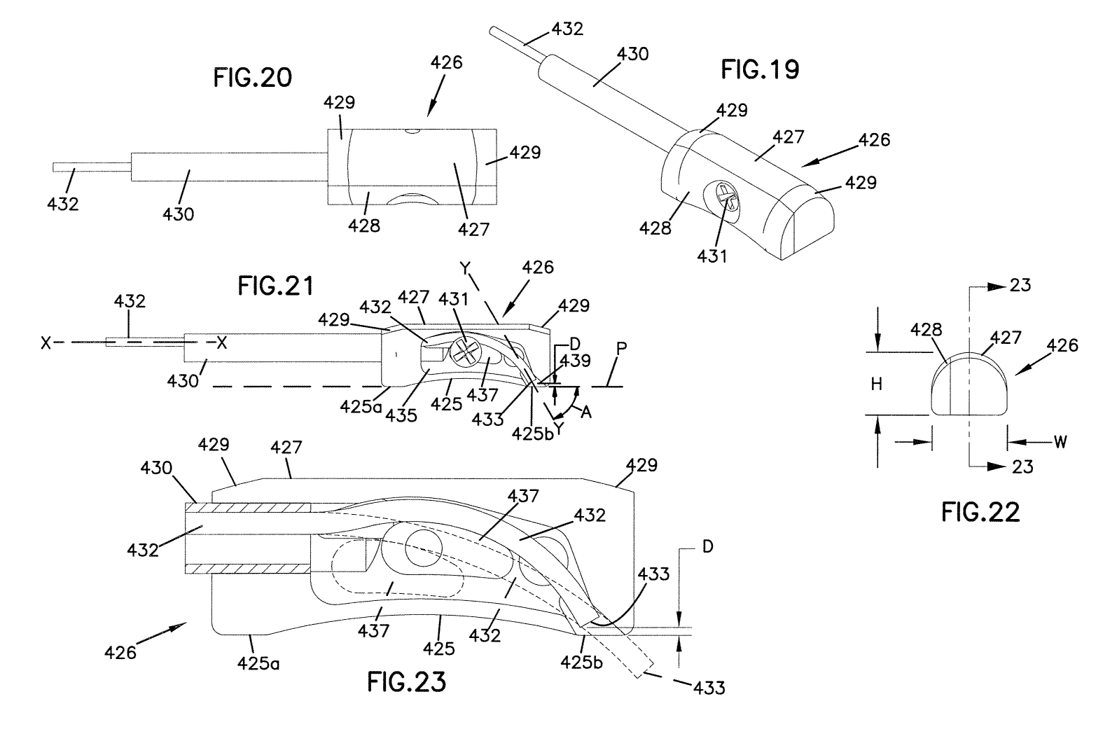

A carriage 426 is provided in the form of a block of biocompatible plastic material such as Delrin® acetal. Other materials (e.g., stainless steel) could suffice. In FIG. 23, a sidewall (shown in FIGS. 19, 20 and 22 as element 428) is removed from the carriage 426 to expose interior components.

The carriage 426 is sized to be slidably received within the interior of the guide member 420. The guide member 420 retains the carriage throughout such sliding motion with the longitudinal axis of the carriage 426 coaxially aligned with the longitudinal axis of the guide member 420.

An upper surface 427 of the carriage 426 is convex rounded and the bottom surface 425 is concave (as best shown in FIG. 21). The upper surface 427 has angled front and rear surfaces 429. This combination of features permits longitudinally sliding movement of the carriage 426 within the guide member 420 when the guide member is bent as illustrated in FIG. 18. This combination also avoids relative rotation between the guide member 420 and carriage 426 to maintain a desired orientation of a laser beam through the bottom wall slot 424 to a target tissue. In a preferred embodiment such tissue is atrial tissue for treating atrial fibrillation but could be tissue of a ventricle for other tissue for treating any other condition (e.g. ventricular tachycardia).

The bottom 425 of the carriage 426 is flat to abut the bottom wall 422. The width W (FIG. 22) of the bottom surface 425 and the height H of the carriage 426 are substantially equal to the internal width and height, respectively of the guide member 420. Accordingly, movement of the carriage 426 with the guide member 420 is limited to longitudinal sliding movement. The flat ends 425a (FIG. 21) of the bottom surface 425 of the carriage 426 remains in contact with the bottom wall 422. The carriage 426 is restricted from transverse movement within the guide member 420.

A conduit 430 extends from a proximal end of the carriage 426 and contains an optical fiber 432. The fiber 432 has a smaller diameter than the conduit 430 to permit passing a cooling and flushing fluid through the conduit 430 during operation as described in early embodiments.

With reference to FIG. 23, the optical fiber 432 terminates at a distal tip 433. In FIG. 21, the carriage 426 is shown with a side cover 428 secured in place by a screw 431. In FIG. 21, the side cover 428 is removed with the screw 431 in place to illustrate internal components. The interior of the carriage 426 has a cavity 435 and an internal block 437 to which the screw 431 attaches.

Opposing surfaces of the cavity 435 is a passageway for placing and re-directing the fiber 432. The fiber 432 enters the cavity 435 with an entrance axis X-X substantially parallel to the conduit 430 and parallel to the plane P of the bottom surface flats 425a.

At the discharge tip 433, the fiber 432 projects through a lower slot 439 of the carriage 426 with a discharge axis Y-Y. The axis Y-Y is at a lesser-included angle A to the plane P. Preferably, the angle A is greater than 45 degrees and, more preferably, greater than 60 degrees.

With this embodiment, the guide member 420 can be wrapped around heart tissue (such as an atrial dome surrounding and connected to the pulmonary veins) to completely surround the pulmonary veins and with the bottom surface 422 snuggly abutting the atrial tissue. The carriage 426 can be moved through the guide member 420 (preferably in a reciprocating manner as previously described) and with laser energy emitted as an end-fire laser from discharge tip 433 towards atrial tissue. While a snug abutment of the bottom surface 422 to the target tissue is preferred, it will be appreciated that a small separation (e.g., a few millimeters resulting from surface irregularities or the like) can be tolerated.

Within the chamber 435, the fiber is free to assume a natural repose abutting the defining surfaces of the cavity 435 and the hub 437 without excessive bending. Also, it will be noted that the spacing between the hub 437 and the chamber walls 435 is greater than the thickness of the fiber so the cooling and flushing fluid is free to pass around the fiber 432 and through the slot 439 to wash debris away from the tip 433.

The tip 433 is recessed within the bottom surface flat 425a by a distance D (FIG. 21) The tip 433 is centrally positioned in the bottom surface flat 425a to be aligned with the slot 424 of the guide member 420.

Preferably, the distance D is about 0.10 inch (about 2.54 mm) and is preferably less than a thickness of the bottom surface 422 of the guide member 420. This maintains a spacing between the discharge tip 433 and the atrial tissue. In a preferred embodiment, such spacing is about 0.05 inches (about 1.3 mm) to provide a clearance for flushing fluid to pass between the discharge tip 433 and the atrial tissue. The flushing fluid can be a gas (such as air or carbon dioxide) or a liquid (such as saline).

In addition to cooling and flushing functions, the fluid acts as a continuous laser light transmissive medium between the discharge tip 433 and the atrial tissue. As result, there is no interface of different materials between the discharge tip 433 and the atrial tissue which might otherwise re-direct laser energy.

The radius of curvature of the fiber 432 within the carriage is about 0.25 inch (about 6.4 mm) for a 400-micron and 0.37 numerical aperture fiber coupled to an 810-nanometer diode laser. This radius is selected to be as tight a radius as possible to maintain as low a profile as possible. At 0.25 inch (about 6.4 mm), there is very little loss of laser energy through the fiber. Such a loss progressively increases as the radius becomes tighter.

With the forgoing embodiment, the radius of the fiber 432 is maintained in the curved shape within the carriage 426 at the time of manufacture. Alternatively, the hub 437 could be a two-position hub such that it can be moved to a downward position in a relaxed state relieving the curvature on the fiber and then moved to the position of FIG. 21 to create the curvature. As a result, the fiber would experience less tension during storage and reduce the possibility of creepage or fracture of the fiber over time when it is in storage awaiting use. Such a position is shown in phantom lines in FIG. 23 with the hub 437 slidably moved downwardly and rearward and the radius of curvature of the fiber 432 relaxed. While a sliding motion is shown, the hub 437 can pivot downwardly in the view of FIG. 23.

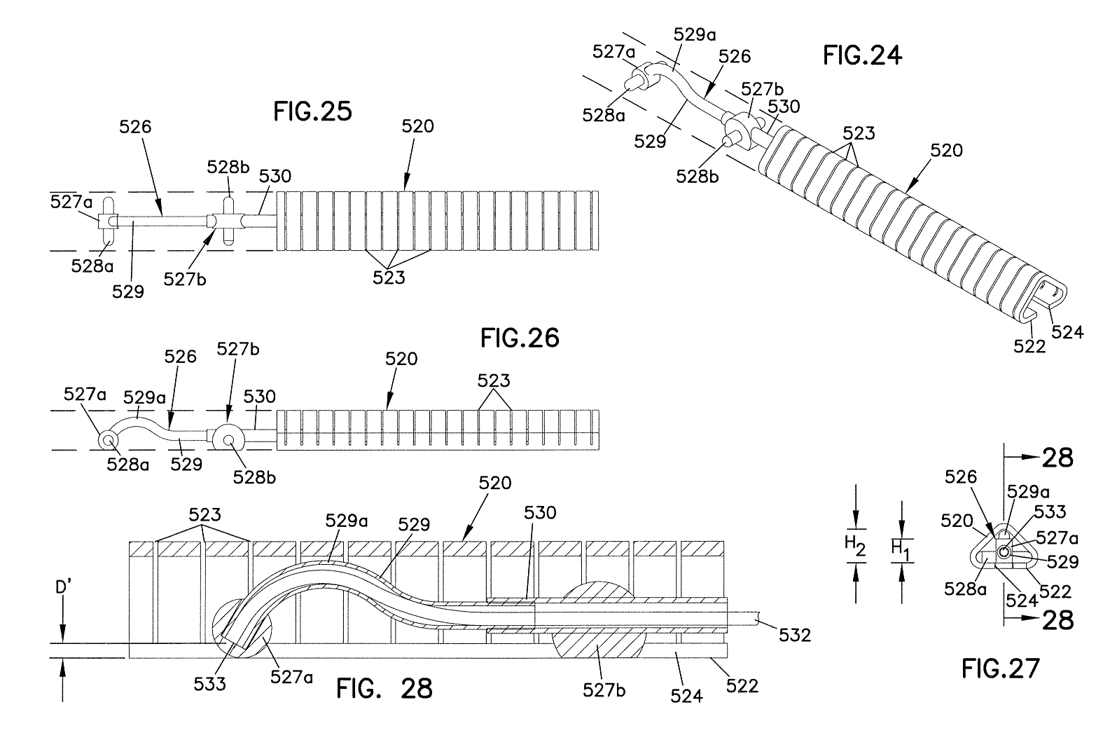

FIGS. 24-28 illustrate an alternative embodiment of a guided end-fire laser fiber for treating tissue such as atrial tissue as described in U.S. patent application Ser. No. 11/228,108. In FIGS. 24-28, the guide member 520 is triangular in cross section and has a base 522 with a slot 524 extending therethrough along the axial length of the guide member 520.

The guide member 520 has slits 523 formed through all sides other than the base 522 and with the plane of the slits 523 substantially perpendicular to the longitudinal axis of the guide member 520. By reason of the slits 523, the guide member 520 can bend around the bottom surface 522 but is resisted from bending in other directions as well as from being twisted.

A carriage 526 includes a distal guide hub 527a and a proximal guide hub 527b. Both of the proximal and distal guide hubs 527a, 527b have extending pins 528a, 528b extending transverse to the longitudinal axis of the guide member 520 (FIG. 27). The phantom lines of FIGS. 24-26 illustrate the carriage 526 resides with the interior of the guide member 620.

The pins 528a, 528b reside near the base 522 to prevent lateral movement of the carriage 526 relative the guide member 520 as illustrated in FIG. 27. The proximal and distal hubs 527a, 527b have a height H₁ smaller than a distance H₂ from the bottom surface 522 to the opposing apex of the triangular guide member 520 (illustrated in FIG. 27).

The carriage 526 also includes a rigid tube 529 connecting the hubs 527a, 527b. The tube 529 is bent upwardly at 529a for the upper end of the bend 529a to abut the apex of the triangular guide member 520 opposing the bottom surface 522. This abutment together with the pins 528a, 528b permits the carriage 526 to move slidably along the longitudinal axis of the guide member 520 but not move laterally or up and down within the guide member 520. Also, this bend represents a maximum radius of curvature of a contained fiber to avoid excessive bending of the fiber.

A conduit 530 is connected to the proximal hub 527b in fluid flow communication with the tube 529. An optical fiber (only the distal tip 533 of which is shown in FIG. 27) may be passed through the conduit 530 into the tube 529 and out through a distal end of the tube 529 to project light through the slot 524 to atrial tissue at an angle preferably greater than 45 degrees and still more preferably greater than 60 degrees. The fiber has a smaller diameter than the interior diameter of the tube 529 and the conduit 530 for flushing fluid to be passed through the tube 529 and around the fiber and past the distal tip 533 to provide cooling and flushing as previously described.

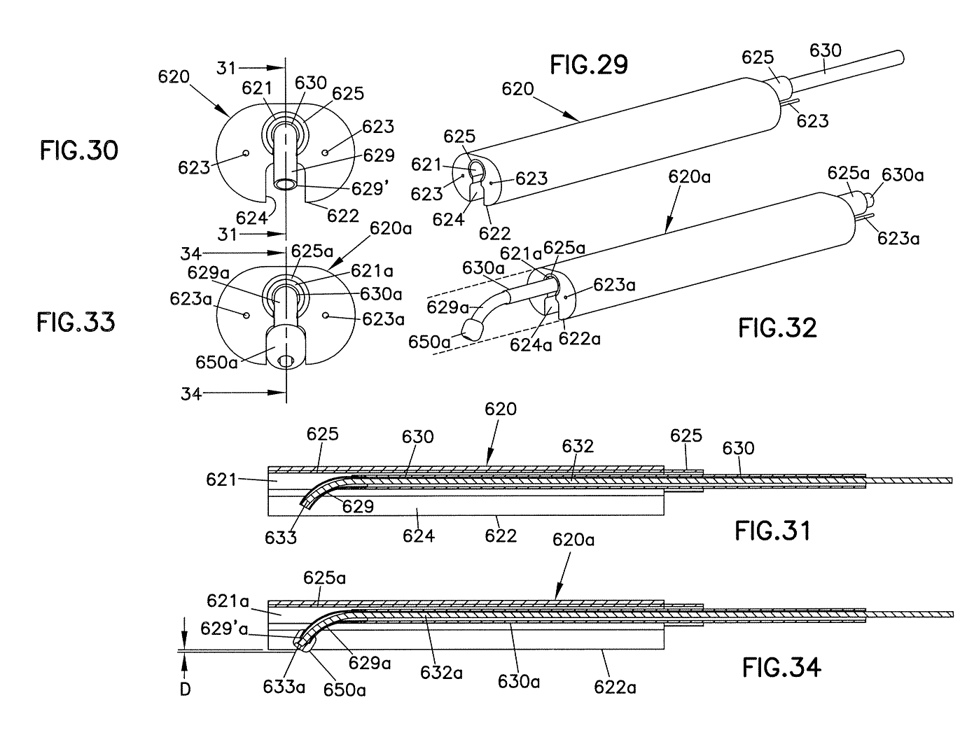

FIGS. 29-34 illustrate a further embodiment according to the '108 application. In these figures, a guide member 620 is shown formed of a highly flexible, biocompatible material (such as extruded or molded silicone, or e-PTFE). A bore 621 is formed throughout the length of the guide member 620. The axis of bore 620 is parallel to the longitudinal axis of the guide member. An arcuate bottom surface 622 of the guide member 620 has a slot 624 extending through the length of the guide member 620 and in communication with the bore 621.

Silicone is highly flexible and easily stretched. To resist stretching in the longitudinal direction, flexible metal cables 623 are molded within the guide member 620 extending through its length on opposite sides of the bore 621. To resisting twisting about the longitudinal axis while permitting bending, a split sleeve 625 of polytetrafluoroethylene (PTFE) is molded to the silicone of the guide member 620 within the bore 621 and with the split of the sleeve 625 aligned with the slot 624. PTFE is also more lubricious than silicone for advantages that will be apparent.

A conduit 630 (made, for example, of flexible, thin-walled stainless steel which is flexible but resists stretching) is slidably received within the bore 621. The annular portion of the bore 621 between the opposing surfaces of the PTFE liner 625 and the conduit 630 permits free longitudinal sliding of the conduit 630 within the bore 621. The PTFE liner 625 permits bending in all directions but resists twisting.

A curved tube 629 is secured to a distal end of the conduit 630. A distal end 629′ of the tube 629 resides within the slot 624. The spacing of the distal end 629′ from the sidewalls of the slot 624 may be narrowed to restrict rotation of the tube 629 and conduit 630 about the longitudinal axis of the guide member 620 while permitting free sliding movement.

An optical fiber 632 as described in the previous embodiments resides within the conduit 630 and moves longitudinally therewith. The diameter of the fiber 632 is smaller than the internal diameter of the conduit 630 to permit flushing and cooling fluid to flow through the conduit 630 as described in earlier embodiments. The conduit 630 and tube 629 act as a guide carriage to direct the fiber distal tip during operation.

A distal tip 633 of the fiber 632 terminates within tube 629 near distal end 629′. The tube 629 redirects the fiber 632 from an entrance axis parallel to the guide member's longitudinal axis to a discharge axis. The discharge axis at the fiber distal tip 633 is as described in earlier embodiments as is the spacing of the tip 633 from the guide member bottom wall 622.

FIGS. 32-34 illustrate an embodiment similar to that of FIGS. 29-31. Similar elements are similarly numbered with the addition of an apostrophe to distinguish embodiments. Except were needed to describe differences between the embodiments, such similar elements are not separately described. In FIG. 32, phantom lines are added to illustrate the tube 629a is intended to reside in the guide member 620a.

The embodiment of FIGS. 32-34 differs from that of FIGS. 29-31 by the addition of a guide tip 650a to the distal end 629a′ of the tube 629a. The guide tip 650a may be identical to that disclosed in commonly assigned U.S. patent application Ser. No. 10/975,674 filed Oct. 28, 2004 (published May 5, 2005 as U.S. patent application Publication No. US 2005/0096643 A1 and incorporated herein by reference). The slot 624a is sized so the side walls of the slot 624a permit sliding movement of the guide tip 650a in the slot 624a while restricting rotation of the tube 629a and conduit 629 about the longitudinal axis of the guide member 620.

Unlike the embodiment of FIGS. 29-31 (in which no element projects beneath the bottom wall 622), the guide tip 650a projects beneath the bottom wall 622a by a spacing D of about 1.0 to 2 mm in a preferred embodiment. The fiber 632a is longer than the fiber 632 but the distance from the distal tip 633a to the bottom lowest projection of the guide tip 650a is preferably the same as distance D′ described with reference to FIG. 28.

With the design of FIGS. 32-34, the guide tip 650a slides atraumatically over the tissue surface while maintaining desired spacing of the fiber distal tip from target tissue.

b. Disclosure of Ser. Nos. 11/385,316; 11/385,317 and 11/385,358

In operating a guided ablation apparatus during a minimally invasive procedure on the heart, the surgeon cannot visually inspect the positioning and movement of the ablation element relative to the heart surface. Instead, the physician can only note the extent to which a proximal end of the apparatus has been pushed or pulled. For example, if the proximal end is pushed five centimeters, the surgeon needs to reliably know that the ablation element has been moved five centimeters.

In the design of a guided ablation apparatus, there may be a possibility of slack or other design characteristics resulting in the ablation element residing in a relatively fixed position even though a proximal end of the apparatus is being manipulated by the position. In such event, it may be possible that an excess amount of energy is applied to a specific location of the heart.

Parent applications Ser. Nos. 11/385,316; 11/385,317 and 11/385,358 are to a design of an ablation apparatus that ensures a closer approximation to a one-to-one unit movement of a distal ablation element in response to movement of a proximal end and further include other safety controls.

With initial reference to FIG. 1, a proximal portion of a guided ablation apparatus 10 is shown in side elevation and with a sidewall partially removed to reveal selected internal components. A distal portion of the apparatus 10 is shown in FIG. 6.

The apparatus 10 includes a flexible guide member 12 and a flexible positioning cord 14 extending from a distal end 16 of the guide member 12. The guide member 12 can be formed of any flexible polymer which can withstand the rigors of sterilization and which is biocompatible for acute use in the human body. By way of non-limiting example, the guide member 12 may be formed of PTFE.

The cord 14 may be formed to be biased to a curved configuration to facilitate placement of the apparatus 10 on a heart. The cord 14 may be releasably attached to the distal end 16 for removal of the cord 14 from the guide member 12. An outer tube 18 extends from the proximal end 19 of the guide member 12.

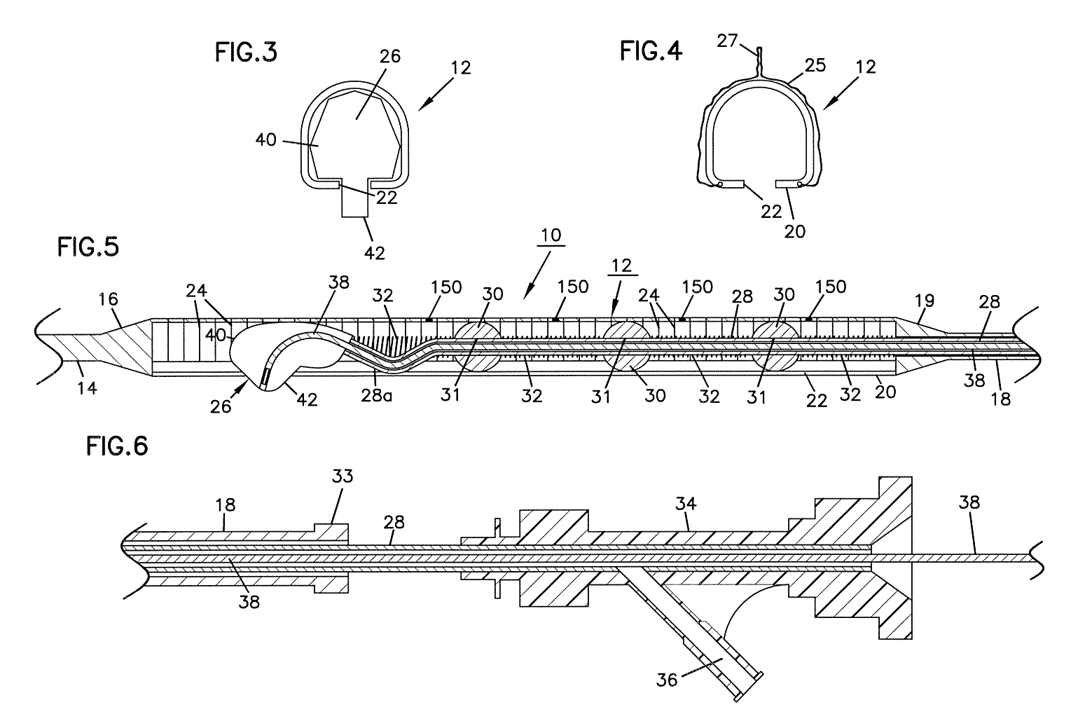

As shown in FIG. 3, the guide member 12 is generally semi-cylindrical in cross section. The guide member 12 has a flat bottom surface 20 with a centrally positioned slot 22 (FIGS. 2 and 3). In a preferred embodiment, the guide member is formed of PTFE (polytetrafluoroethylene).

The sidewalls and top of the guide member 12 have a plurality of transverse slits 24 along the length of the guide member 12. The plane of each slit 24 is perpendicular to the longitudinal axis of the guide member 12. The slits 24 do not extend through the bottom surface 20.

FIG. 4 illustrates an alternative embodiment where the outer surface of the guide member 12 (except, in a preferred embodiment, the bottom surface 20) is covered with a flexible covering material such as fabric 25. The fabric 25 is preferably PTFE or ePTFE, which has a longitudinal stretch to permit it being bent with the guide member 12. The covering material 25 may also be sheet material such as silicone. The fabric 25 is attached to the guide member 12 (e.g., by adhesion, heat staking or the like). The fabric 25 acts as a covering over the slits 24 to prevent body fluids or debris from migrating into the interior of the guide member 12 and possibly interfering with internal components. The covering 25 also prevents the slits 24 from snagging on tissue. Further, the fabric 25 may be provided with a plurality of tabs 27 to permit a surgeon to easily grasp the guide member 12 by forceps. If desired, markers (not shown and which may be radiopaque) can be placed on the covering 25 or guide member 12 to identify any location or segment of the covering 25 or guide member 12.

By reason of the slits 24, the guide member 12 is highly flexible. As shown in FIG. 7, the guide member 20 may be straight. Due to the slits 24, the guide member 12 may be bent downwardly (FIG. 1A). While not desirable, the slits 24 and flexibility of the guide member 12 may permit it be bent slightly to the left or the right as illustrated in FIG. 8 or twisted as in FIG. 9. However, it is preferred such left or right bending or twisting be minimized. “Left” or “right” are relative terms with the bottom surface 20 of the guide member 12 being defined as “down” and the distal end 16 being defined as “front”.

Be reason of its flexibility, the guide member 12 may be placed over the heart surface in a desired Maze pattern and with the bottom surface 20 opposing the heart's epicardial surface.

In placing the guide member 12, a surgeon can grasp and pull the cord 14 to manipulate the distal end 16. The guide member 12 may be provided with an intermediate connector 160 (schematically shown in FIGS. 1A and 1B) to permit the distal end 16 of the guide member 12 to be bent around and attached to the connector 160 such that the guide member 12 forms a loop. This permits placement of the guide member 12 completely encircling pulmonary veins and define a complete pathway around the pulmonary veins for ablation.

The guide member 12 houses a follower or carriage 26. A flexible inner tube 28 (shown in FIG. 5) is connected to the carriage 26. The inner tube 28 extends proximally from the carriage 26 throughout the length of the guide member 12 and through the tube 18. In the presently preferred embodiment, carriage 26 is formed of stainless steel.

A plurality of Teflon® spacer spheres 30 reside in the guide member 12 between the proximal end 19 and the carriage 26. Each of the spheres 30 has an axial bore 31 (FIG. 5) which receives the inner tube 28 such that each sphere 30 is freely slidable on the tube 28. In the schematic representation of FIG. 8, only three spheres 30 are shown for ease of illustration.

A plurality of springs 32 extends between each opposing sphere 30. Further, a spring 32 extends between the most distal sphere 30 and the carriage 26. Also, a spring 32 extends between the most proximal sphere 30 and the proximal end 19 of the guide member 12.

As an alternative to a plurality of springs 32, a single spring can extend from the proximal end 19 to the carriage 26. In such embodiment, the bores 31 of the spheres 30 are sized to surround the spring so the spheres 30 can slide axially relative to the spring. Also, with a single spring, the spheres 30 can be eliminated and be replaced by providing the spring with a plurality of spaced apart, enlarged diameter portions.

As shown in FIG. 6, the outer tube 18 terminates at a proximal end which includes a coupling 33. The inner tube 28 extends slidably through the coupling 33 and terminates at a Y-connector 34. A fluid inlet 36 of the Y-connector 34 permits injection of a fluid into the tube 28. The tube 28 houses an optical fiber 38 which extends beyond the connector 34 and runs throughout the length of the inner tube 28, tube 28a and into the carriage 26.

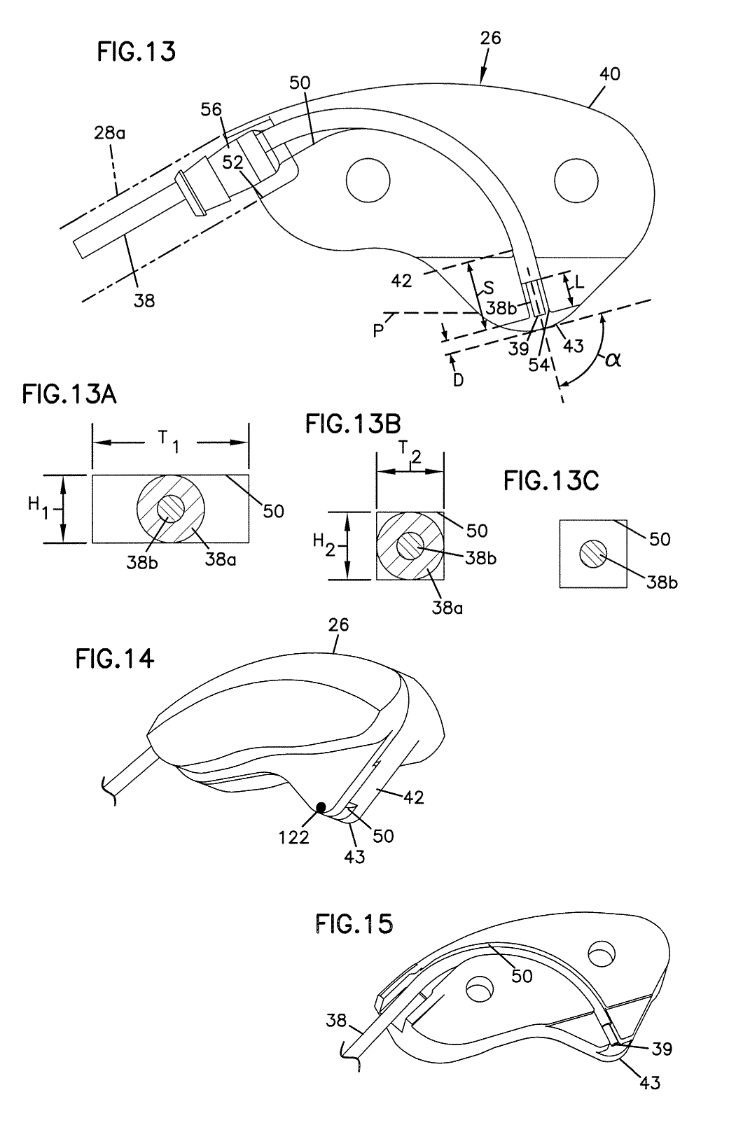

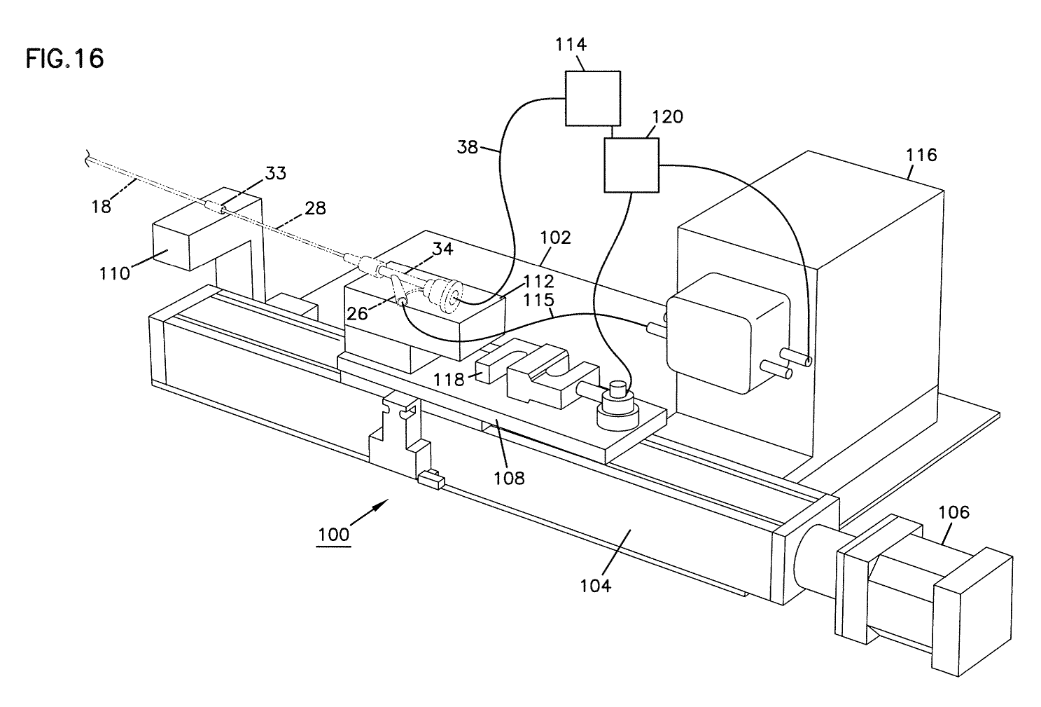

With reference to FIGS. 13-15, the carriage 26 is shown in greater detail. FIGS. 13 and 15 show the carriage 26 with a right side panel removed to expose interior elements.

The carriage 26 includes a main body 40 which is sized to be received within the interior of the guide member 12. A bulge extension 42 is sized to be received within the slot 22.

As shown in FIG. 3, the geometry of the carriage 26 is selected such that it mates with the interior surface of the guide member 12 to permit sliding movement of the guide member along the longitudinal axis of the guide member 12 but preventing relative rotation of the carriage 26 and the guide member 12. Further, the amount of protrusion of the bulge 42 beyond the bottom surface 20 is preferably fixed. Accordingly, when the bottom surface 20 of the guide member is placed abutting the tissue of the heart, the bulge portion 42 presses slightly into the tissue to ensure contact with tissue of the heart throughout movement of the carriage 26.

The carriage 26 has an internal channel 50 sized to receive the optical fiber 38 and direct the optical fiber 38 from an inlet end 52 to an outlet end 54. At the inlet end 52, a connector 56 connects the tube 28 (shown in phantom lines in FIG. 13) with the carriage 26 and permits the optical fiber 38 to pass through the inlet 52 towards the outlet 54.

When moving the carriage 26 over a heart surface, the tissue surface may be irregular or, due to bending, the guide member 12 may be slightly spaced from the tissue. To maintain a constant spacing between tissue to be treated and the fiber tip 39, the bulge 42 is provided. Constancy of spacing controls the energy applied to tissue as well as controlling a layer of cooling fluid between the tissue and the fiber tip 39.

The bulge 42 has a farthest protrusion 43. The optical fiber 38 is held by the carriage 26 with the fiber tip 39 spaced from the furthest protrusion 43 by a distance D. Preferably, the length of distance D is about 0.5 mm. As a result, the fiber tip 39 is always maintained in a spaced distance from the tissue of the heart during an ablation procedure. A length L of the fiber 28 at tip 39 has the outer jacket of the fiber 28 removed to limit incidences of reflected energy flash back. In the preferred embodiment, the length L is 1.25 mm.

The prior publications and parent application describe the fiber connected to a laser source having wavelength ranges of 790 nm to 830 nm or 1020 nm to 1140 nm with a preferred wavelength of about 810 nm. Such wavelengths are preferred for their characteristic low water absorption. As used herein, low absorption means less than 30%. While any wavelength in such range is suitable in a preferred embodiment, a most preferred wavelength is 1064 nm. While an 810 nm wavelength is acceptable, Applicants have found that, in addition to low water absorption, a 1064 nm wavelength also exhibits low absorption in myocardial tissue when directed at such tissue from the epicardial surface of the atrium. Also, such wavelength is readily producible through commercially available lasers (such as Nd:YAG lasers). This wavelength also exhibits low surface reflectivity resulting in reduced thermal stress on the fiber 38. Also, a YAG laser permits use of a smaller fiber with a tighter bending radius in carriage 26 which can result in a lower profile guide member 12.

The pathway 50 has a straight portion S near the outlet 54 with the remainder of the passage 50 being curved. With this geometry, the optical fiber 28 extends coaxially with the axis of the guide member 12 as is bent such that the tip 39 discharges laser energy toward the heart tissue. As in the parent application, a low profile ablation apparatus is provided with the benefits of an end-fire laser fiber 28.

In FIG. 13, the plane of the heart tissue is shown in phantom lines as P. The angle between the plane P and the axis of the fiber discharge is shown as α. While α can be 90 degrees, in a preferred embodiment it is about 72 degrees for use with a 400 micron fiber operating as an 1064 nanometer laser energy wavelength.

The length of the straight segment S is selected to avoid overlapping the regions of mechanical stress and thermal stress on the fiber 28 during operation. Namely, laser energy may be reflected off of the heart tissue back into the laser tip 39. The reflected laser energy imparts a thermal stress to the optical fiber 28. Further, the bending of the fiber 28 within the channel 50 imparts a mechanical stress to the fiber 28.

In order to minimize the total stress on the fiber 28, it is desirable that the area of appreciable thermal stress not overlap with the mechanical stress. The straight segment S does not impart a mechanical stress to the fiber 28.

The length of the straight segment S is selected such that the thermal stress from reflected laser energy is substantially dissipated throughout the length S. The straight length is a function of the fiber size, angle α, the numerical aperture of the fiber, the operating wavelength and the power, the distance D and surface reflectivity of target tissue. In a treatment for atrial fibrillation as described, and using a 400-micron fiber operating at 1064 nanometers with a maximum power of 25 watts and an angle of 72 degrees and numerical aperture of 0.22, this straight length is preferably greater than 0.100 inch (about 2.54 mm).

Since the tube 28 is hollow and may receive a cooling fluid as previously described, a cooling fluid may be flushed through the channel 50 and around the fiber 38 and through the outlet 54 against the tissue during the ablation process for benefits previously mentioned. The cross-sectional geometry of the channel 50 varies throughout its length. In the region of the curved portion of the fiber 38, the channel 50 has a rectangular cross-section illustrated in FIG. 13A. The transverse dimension T (which extends in a direction perpendicular to the sidewalls of the guide member) is greater than the diameter of the outer jacket 38a of fiber 38. The height H₁ (i.e., the dimension perpendicular to dimension T₁) is approximately equal to the diameter of jacket 38a. With this geometry, coolant flows along the sides of the fiber 38. In the region of straight segment, the cross-section is square (FIG. 13B) with both dimensions H₂ and T₂ equal to the diameter of jacket 38a (H₁ equals H₂). The coolant now flows through the much smaller area in the corners of the channel 50 thereby increasing the velocity of coolant flow. In the length L (with the jacket 38a removed to expose the fiber core 38a), the cross-section (FIG. 13C) of the channel 50 remains the same as in FIG. 13B but the coolant can now flow completely around the fiber core 38a. A preferred flow rate past the discharge tip 39 is about 10 ml/minute.

With the structure thus described, by pushing on the connector 34 and moving it toward connector 33, the inner tube 34 and fiber 38 are moved distally relative to the outer tube 18. Further, the motion of the tube 28 imparts a distal movement of the carriage 26 relative to the guide member 12. Throughout this movement, laser energy may be applied through the laser tip 39 to ablate tissue.

Since the guide member 12 may be curved or twisted in a wide variety of geometries, it is possible (but for the structure described herein) that the tube 28 can become curved within the guide member 12. If such were to occur, movement of the connector 34 would not necessarily result in corresponding movement of the carriage 26. Accordingly, a physician could conclude erroneously that the apparatus was applying laser energy uniformly over a length of the heart tissue when, in fact, all of the energy is being applied to a single location on the heart tissue which could result in excess heating of that location.

The invention of parent applications Ser. Nos. 11/385,316; 11/385,317 and 11/385,358 reduces this undesirable effect by reason of the spheres 30 and springs 32. The spheres 30 maintain the tube 28 axially positioned within the guide member 12. Equal spacing of the spheres 30 along the length of the guide member 12 is provided by the springs 32.

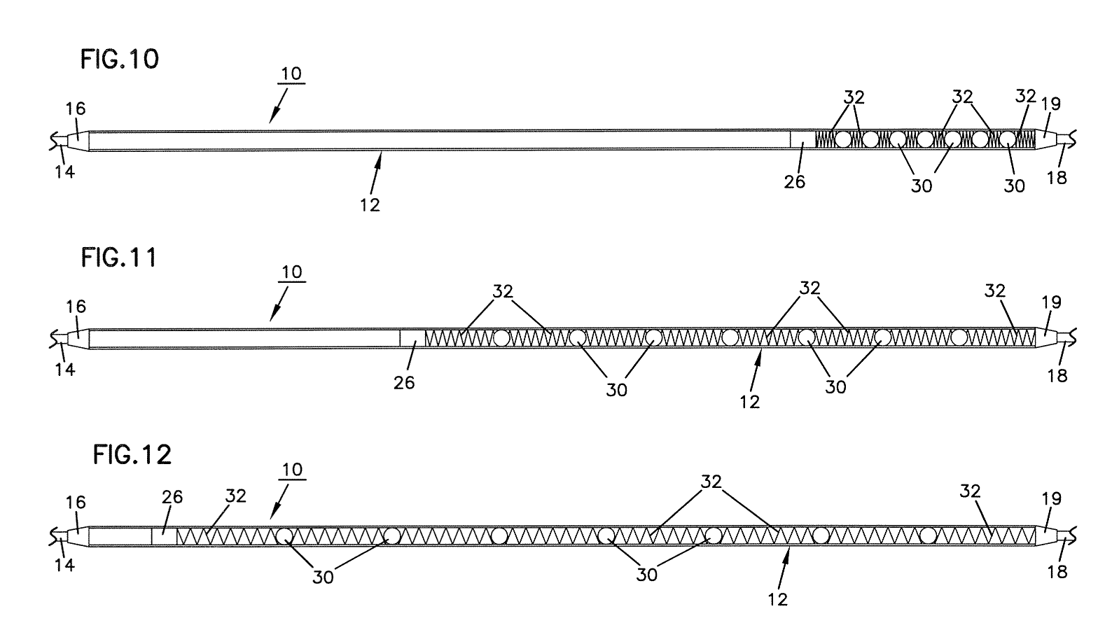

FIG. 10 illustrates, in schematic format, the apparatus when the carriage 26 is near the proximal end 19 of the guide member 12. The springs 32 (which are equal in length) are fully compressed and maintain the spheres 30 equally positioned along the length of the guide member 12. The spheres 30 maintain the inner tube 28 axially positioned within the guide member 12. At the carriage 26, the tube 28a is free to bend as illustrated by bent segment 28a in FIG. 1.

FIG. 11 illustrates the apparatus 10 with the carriage 26 moved partially away from the proximal end 19 of the guide member 12. As a result, the springs 32 relax and expand equally. In a preferred embodiment, the springs 32 are of equal length and have equal spring constants. Since the springs 32 expand equally, the spacing between the spheres 30 is increased but maintained constant for each of the spheres 30. As the carriage 26 is further extended (FIG. 12), the springs 32 are shown in an almost completely expanded configuration yet maintaining equal spacing between the spheres 30.

FIG. 16 illustrates a control unit 100 for the present apparatus. In FIG. 16, various components are shown schematically for ease of illustration. A control unit 100 includes a stationary platform 102 on which is mounted a linear actuator 104 connected to a stepper motor 106. The linear actuator may house a threaded rod (not shown) or the like driven by the motor 106.

A mounting plate 108 is carried on the actuator 104 such that it moves in a linear path in response to rotation of the stepper motor 106. A fixed mount 110 is connected to the platform 102. The mounting plate 108 carries a catheter mount 112 aligned in the same plane as the fixed mount 110. Accordingly, as the stepper motor 106 is rotated, the catheter mount 112 moves toward or away from the fixed mount 110.

The coupling 33 may be fixed to the fixed mount 110 through any suitable means and the connector 34 fixed to the moving mount 112. Such positioning is shown in phantom lines in FIG. 16. Accordingly, as the mounting plate 112 is moved towards the fixed mount 110, the carriage 26 is moved distally within the guide member. As the moving mount 112 moves away from the fixed mount 110, the carriage 26 is moved proximally within the guide member.

The fiber 38 extends to a power source 114 with enough slack and excess length in the fiber 38 to accommodate movement of the mounting plate 112 toward and away from the fixed mount 110. The fluid inlet 36 is connected via a line 115 to a pumped fluid source 116 for delivery of a cooling fluid into the tube 28 as described. If desired, a bubble trap can be included to avoid airflow into the tube 28.

The moving mount 112 is connected to a load cell 118 which acts as a sensor to sense the amount of force being applied to the mount 112. As a further sensor, the amount of reflected laser energy reflected back through the fiber 28 can be measured by a monitor 120 as known to one of ordinary skill in the art. Further, the amount of force measured by the load cell 118 can also be directed to monitor 120 and the flow rate and coolant fluid pressure at the pump 116 can be measured and displayed by monitor 120.

As a result, an operator can measure the amount of force applied to movement of the carriage 26 within the guide member 12. If the force exceeds a predetermined minimum, the operator may presume that, for whatever reason, the follower or carriage 26 is snagged within the guide member 12 and laser energy may be stopped to prevent overheating the tissue.

A further sensor is provided in the form of a thermocouple 122 (shown schematically in FIG. 14) positioned on the carriage near protrusion tip 43. When laser energy is being applied, the thermocouple detects a rise in temperature. If the laser is energized and no rise in temperature is detected, an operator may assume there is a break along the length of fiber 28. Also, since the thermocouple 122 is positioned near tissue to be treated, the thermocouple 122 can detect excessive heat indicating the tissue may be approaching undesired carbonization temperatures. In such event, the controller 100 discontinues laser energy if the sensed temperature exceeds a pre-determined maximum (e.g., exceeds a target of between 80° C. and 100° C.).

Reflection displayed by monitor 120 may also provide an indication of overheating of tissue. As tissue overheats, it can carbonize resulting in blackened tissue. Such blackened tissue has a different reflectance than tissue which has not been carbonized. The difference in reflection is displayed on the monitor 120. If the monitor 120 indicates carbonization, laser energy can be discontinued. Also, the amount of fluid flowing to the tube 28 can be measured. If the flow or coolant pressure falls below a desired minimum (which could result in overheating of the fiber as well as overheating of the tissue being treated by the fiber), laser energy can be discontinued.

In addition to the controls thus described, movement of the carriage 26 within the guide member 12 can be measured by any suitable means. For example, sensing elements 150 (such as Hall-effect transistors) (FIG. 5) can be spaced along the length of the guide member 12. Such sensors 150 detect movement of a ferro-magnetic carriage 26 past each of the sensors 150. In addition to providing an indication of positioning of the carriage 26 within the guide member 12, the time-based derivative of movement of the carriage 26 past the sensors 150 can indicate speed of travel of the carriage 26 within the guide member 12. Other motion detection techniques include an electrically resistive element in the guide member 12 with the carriage 26 completing a circuit with the total resistance being a function of displacement of the carriage 26 within the guide member 12. Also, optical sensing can be used.

C. Additional Disclosure of the Present Application

Notwithstanding the improved design described with reference to FIGS. 1-17, a movement of a proximal end of the fiber 28 may still vary from an actual movement of the distal fiber tip 39. When pushing or pulling the proximal end of the fiber 28 at connector 34 in FIG. 16, the proximal end may move a distance before the tip 39 starts to move. This distance (referred to as “hysteresis”) may vary from time to time depending on the position of the fiber tip 39 in the guide member 12, the amount of twisting or bending of the guide member 12 and other factors.

By testing any given design, the maximum amount of hysteresis can be noted. For example, if such testing reveals a maximum hysteresis of 0.5 cm, a more conservative maximum (e.g., 1 cm) can be assumed with confidence the actual hysteresis will not exceed the assumed maximum hysteresis during use of the device on a patient. While the maximum hysteresis may vary from design to design, the principles of the present invention remain the same.

Hysteresis results in the fiber tip 39 remaining stationary over a tissue location at the initiation of the proximal end of the fiber 28 being moved through the hysteresis distance. After such movement, the fiber tip moves in an amount corresponding to the remaining movement of the fiber proximal end.

If the fiber were energized during the period of hysteresis, excessive energy could be applied to the tissue location over which the tip 39 resides during the period of hysteresis. Too much energy applied to this site can result in undesired carbonization or perforation of the tissue.

With an assumed maximum hysteresis, the application of energy to a tissue site can be controlled. Namely, during such period of assumed hysteresis, the laser power source can be turned off. As a result, laser energy is not applied to the tissue surface over which the fiber tip is stationary during such hysteresis period.

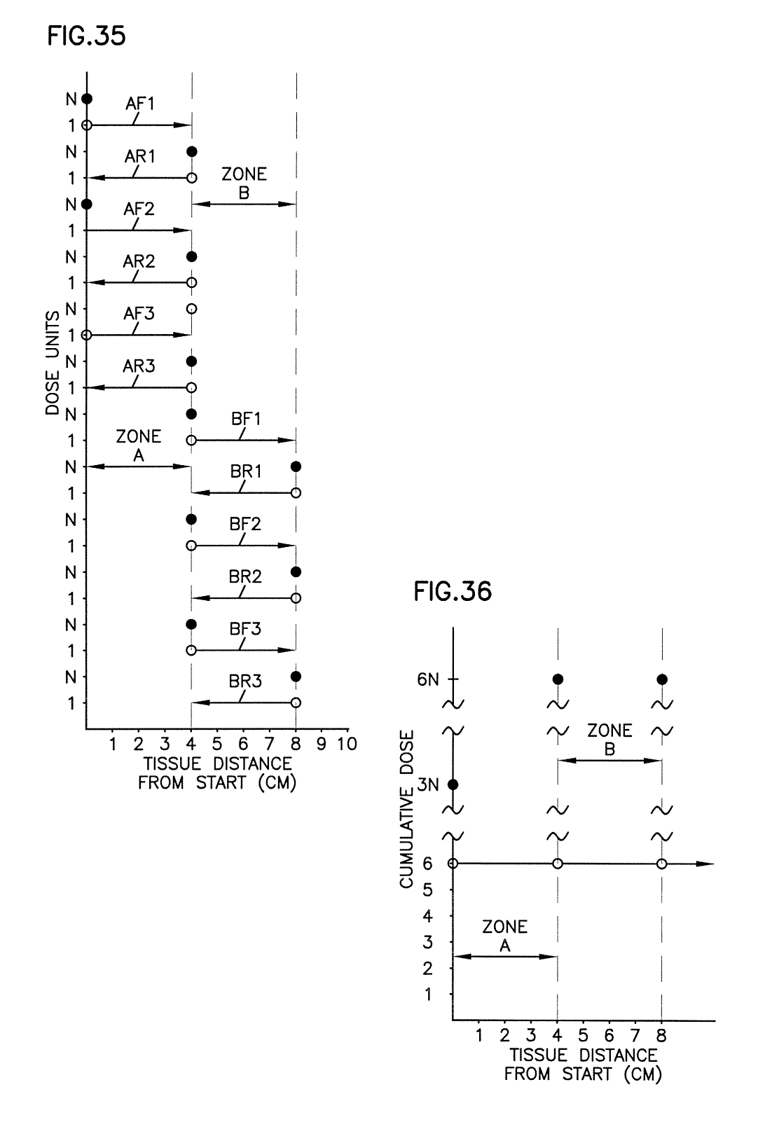

With respect to prior applications noted above, a then preferred embodiment was noted of dividing the targeted lesion pathway into a plurality of zones. Within a zone, the laser is energized and the laser tip 39 is moved back and forth within the zone. It has since been determined the use of a zone application presents risks of perforation or non-transmural (under-treated) lesions. This is illustrated with reference to FIGS. 35 and 36.

When creating a MAZE pattern lesion on the epicardial surface of an atrium, an amount of desired dosage of laser energy is determined. The amount of laser energy to be applied to the heart tissue is a function of a number of factors. These include the power (usually measured in watts) of the laser, the laser wavelength, the spacing and angle of the laser tip and the speed at which the laser tip moves over the tissue.

Anatomical variances may effect dosing. For example, minor variations in tissue surface geometry and tissue make-up (e.g., fat layers over the epicardium) can impact energy absorption. Also, the atrium is filled with blood which is being pumped by the heart. Blood flow in the atrium can cool the atrial tissue between dose applications. The amount of cooling will vary with the time between doses.

For ease of illustration, certain factors will be assumed for illustrating the present invention. Namely, the laser source is a 1064 nm Nd:YAG laser with an operating power of 25 watts. The laser tip spacing and orientation and guide mechanism are as described with reference to FIGS. 1-17.

The speed of travel of the laser tip in the guide member is 3 mm/second. The deceleration and acceleration of the laser tip are assumed to be instantaneous and the power up and power off of the laser is assumed to be instantaneous. Of course, these assumptions are not possible. However, in practice, these assumptions are very close to reality. Acceleration and deceleration are very rapid as is the power up and power off of the laser such that these assumptions are reasonable approximations. For applications where these assumptions are deemed inadequate, one of ordinary skill in the art with the benefit of the teachings of the present invention can readily modify the present method.

In testing an apparatus made according to FIGS. 1-17, a maximum hysteresis of about 0.5 cm has been noted. Therefore, an assumed maximum hysteresis of 1.0 cm is conservatively assumed to create an added safety factor. In the description of the preferred embodiment, hysteresis is assumed to occur only in a push direction (i.e., moving the fiber tip 39 distally within the guide member 12). With the benefits of the teachings of the present invention, one of ordinary skill in the art can apply the invention to account for hysteresis in the opposite direction or in both directions.

With the laser operating assumptions noted above, a representative target dosage could be six exposures. In other words, tissue to be treated is ideally passed by an energized laser tip six times. A dosage is selected so that more or less dosage is tolerable as long as the variability is not so great that too small of a cumulative dosage would result in a non-transmural lesion or too great of a dosage would present an unacceptable risk of tissue carbonization or perforation.

FIGS. 35 and 36 illustrate the problems of treating tissue in zones without accounting for hysteresis. In the example of these figures, a target zone length is 5 cm. The laser tip starts energizing at location “0”. The intended first zone (Zone A) is targeted to end at tissue location 5 cm from the start. At this point, a second zone (Zone B) is intended to start. Zone B is intended to end at a tissue location 10 cm from the starting point.

The laser tip is moved over the tissue in Zone A six times. This results in three forward passes (arrows AF1, AF2 and AF3) and three rearward passes (AR1, AR2 and AR3). After completion of the last rearward pass (AR3) in Zone A, the fiber tip is moved by advancing the proximal end of the fiber 5 cm. In Zone B, the treatment process is repeated with three forward passes (arrows BF1, BF2 and BF3) and three rearward passes (BR1, BR2 and BR3). The process is then repeated in a next contiguous zone (not separately shown). In the figures, the direction of the movement of the fiber tip is illustrated by the directional arrows of the passes.

Throughout the entire movement of the fiber tip in any of the passes, the laser is turned on. At intermediate locations of the passes (i.e., between the tail and the tip of the arrows in FIG. 35), the tissue opposed by the fiber tip is subject to one dose unit (number “1” on the vertical axis of FIG. 35).

FIG. 35 illustrates the individual passes over the tissue and resulting applied dosage per pass. FIG. 35 shows the displacement of the fiber tip over tissue as a proximal end of the fiber is moved five centimeters. FIG. 36 illustrates the resulting accumulative applied dosage to the tissue in Zones A and B.

Inspection of FIGS. 35 and 36 results in several observations. First, the Zones A and B are four centimeters in length instead of a targeted five centimeters. As a result of the 1 cm of hysteresis, while the proximal end of the fiber is pushed or pulled at connector 34 a total of five centimeters per pass, the first centimeter of each push and pull results in no motion of the fiber tip. Therefore, the fiber tip only moves four centimeters per pass.

Second, while there is uniform and desired dosage applied between the arrow tails and tips of each pass (i.e., one dose unit per pass), the starting dosage at the tail of the arrow of each pass is much greater. Namely, during the one centimeter of hysteresis, the fiber is energized but the fiber tip is not moving. As a result, for the time required to move the proximal end of the fiber the first centimeter, the laser tip is applying energy to a single spot on the tissue.

In FIGS. 35 and 36, “N” is the dose applied while the fiber tip is stationary during 1 centimeter of hysteresis. As a consequence, the starting point (location “0” in FIGS. 35 and 36) receives a total cumulative dosage of 3N. The junction point between the Zones A and B receives a cumulative dosage of 6N. Any subsequent junction between contiguous zones also receives a dosage of 6N.

Assuming the proximal end is moved at 3 mm/sec and the hysteresis is 1 cm and laser power is 25 W, a 1-second exposure to tissue applies 25 Joules (J). Except at the ends of the zones, the fiber tip is moving uniformly over tissue at 3 mm/sec. Each 1 mm of tissue in this path receives a dose of 8.33 J per pass. Therefore, after six passes, the dosage applied to the tissue (other than at the ends of the zones) is about 50 J.

Due to hysteresis, the dosage at the ends of the zones is much greater. The time elapsed during a 1-centimeter hysteresis is 3.33 sec (10 mm×1 sec/3 mm). Therefore, in FIGS. 35 and 36, N is about 83 J, 3N=249 J and 6N =498 J (almost ten times the desired dosage).

It will be appreciated that dosage described in the foregoing and following examples are for the purpose of illustration. Actual dosage will vary with speed of the fiber tip, spacing of the fiber tip from the tissue and laser power. A desired total dosage will vary depending upon a variety of factors including atrial wall thickness.

In FIGS. 35 and 36, it is assumed the maximum hysteresis (1 cm) is actually experienced at the start of each pass. In fact, such hysteresis can vary considerably with each pass (while staying below the assumed maximum of 1 cm). This can also alter the treatment unacceptably. For example, if the passes of Zone A each experience an actual hysteresis of 1 cm but the movement from pass AR3 to pass BF1 has an actual hysteresis of only 0.5 cm and all passes in Zone B have an actual hysteresis of 0.5 cm, then there will be a 0.5 cm gap of untreated tissue between Zones A and B. Further, passes within a zone can have varying hysteresis resulting in an unacceptable variability in cumulative dosage.

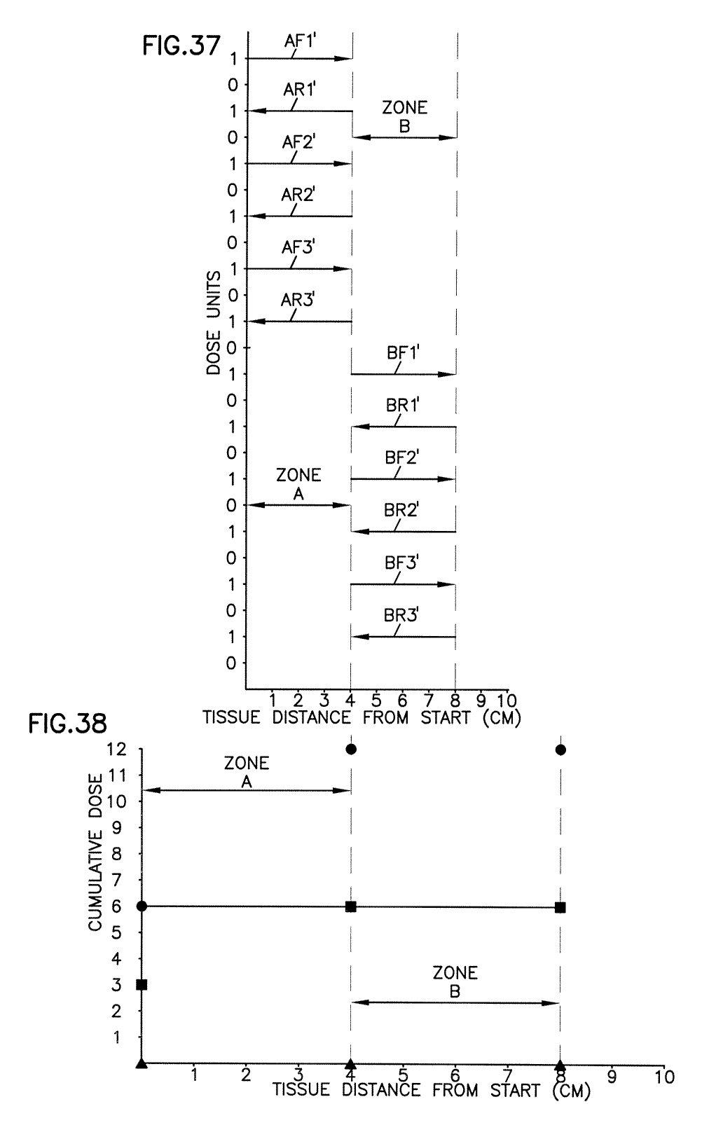

One technique for accounting for hysteresis would be to turn off the power to the laser during periods of assumed maximum hysteresis. During the first 1 cm of travel of the proximal end of the fiber, the laser is off. This avoids the very high dosages (N) of FIG. 35. However, as illustrated in FIGS. 37 and 38, this treatment continues to be unacceptable.

FIG. 37 replaces the forward and rearward passes of FIG. 35 with new forward passes AF1′, AF2′, AF3′, BF1′, BF2′ and BF2′ and rearward passes AR1′, AR2′, AR3′, BR1′, BR2′ and BR3′. In each of these passes, the laser is turned off during the first centimeter of travel of the proximal fiber end and during one during the last four centimeters of travel of the fiber proximal end.

FIGS. 37 and 38 assume a first extreme case where the actual hysteresis per pass equals the assumed maximum hysteresis of 1 cm. As a result, while the power is off, the fiber tip is not moving over tissue. Further, while the power is on, the tip is not stationary over a tissue site. In theory, each location on tissue receives the desired dosage of 6 dose units (about 50 J using the foregoing assumption). Each zone is a uniform 4 cm in length since the proximal end moves 5 cm with the first 1 cm of motion having no corresponding distal tip motion due to hysteresis.

Unfortunately, an inspection of FIGS. 37 and 38 with a consideration of underlying assumptions reveals the method of FIGS. 37 and 38 is unacceptable. One possible variance from theoretical is due to the dosage applied at the start and stop of each pass. For example, due to operating variances in equipment, tissue under the fiber tip at the start of laser power (the arrow tails in FIG. 37) or the stop of laser power (the arrow tips in FIG. 37) can receive one dose (numeral “1” in the vertical axis of FIG. 37) or no dose (numeral “0” in the vertical axis of FIG. 37).

FIG. 38 illustrates the cumulative dosage applied to tissue within Zones A and B. While dosage is a desired 6 doses between the start and stop of the Zones (i.e., other than at locations 4 cm and 8 cm from the start of treatment), dosages at zone boundaries can vary considerable.

The circle data points illustrate the cumulative applied dosage assuming tissue is treated beneath the fiber tip at the instant of power on and off (i.e., both at the tails and tips of the arrow passes). The square data points illustrate cumulative dosage assuming tissue treatment only under the arrow tips. The triangular data points illustrate an extreme case of no treatment under either the tips or tails of the arrows. It will be noted that at the zone boundary (e.g., location 4 cm from the start of treatment in FIG. 38), this can result in a treatment variance between 12 doses (100 J with the example given) to a low of zero doses (also unacceptable).

Also, as in the previous example, FIGS. 37 and 38 assume the maximum assumed hysteresis (1 cm) is the actual hysteresis experienced at the start of each pass. Variances in actual hysteresis can result in zone overlap with a potential for double dosing or zone gaps with zero dosing.

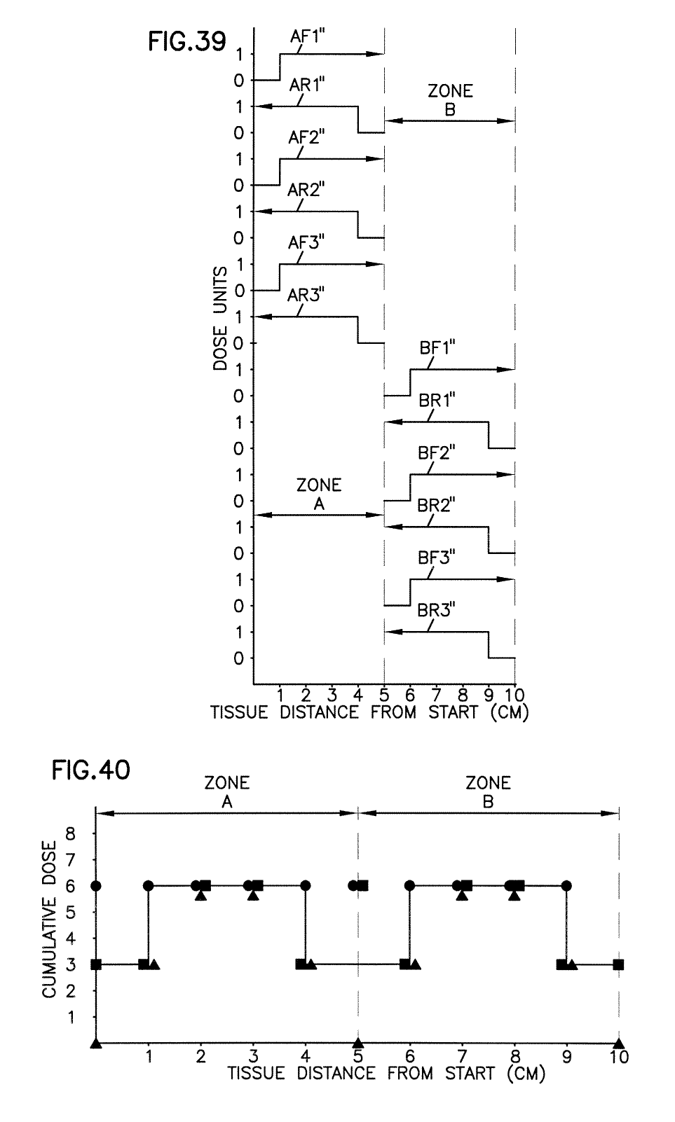

FIGS. 39 and 40 illustrate results if actual hysteresis per pass equals zero. While a zero hysteresis is not likely, it represents an extreme case. Since there is no hysteresis, each pass and each zone is five centimeters long. The first centimeter of travel of the fiber tip results in no dosage (labeled “0” in the vertical axis of FIG. 39) delivery while the last four centimeters of travel delivers one dose unit (labeled “1” in the vertical axis of FIG. 39) at each tissue location. Passes are labeled the same as in FIG. 37 but with a double apostrophe to distinguish the examples.

FIG. 40 illustrates the cumulative effects. The data points (circle, square or triangle) have the same meaning as in FIG. 38.

The potential twelve dose unit spike of FIG. 38 at zone boundaries is avoided. However, a gap of half dosage (three dose units) occurs at the boundary. The acceptability of a 50% reduction from target dosage will vary depending on the target tissue, tissue thickness, etc. The potential for no dose delivery at the boundaries continues. A no dose event is unacceptable since it could result in a lesion with a gap.

Also troubling, FIGS. 39 and 40 assume the actual hysteresis is a constant zero hysteresis per each pass. This is practically impossible. Therefore, variability risks continue between passes and between zones. These can result in unacceptable gaps between zones or double dosage in overlapping zones.

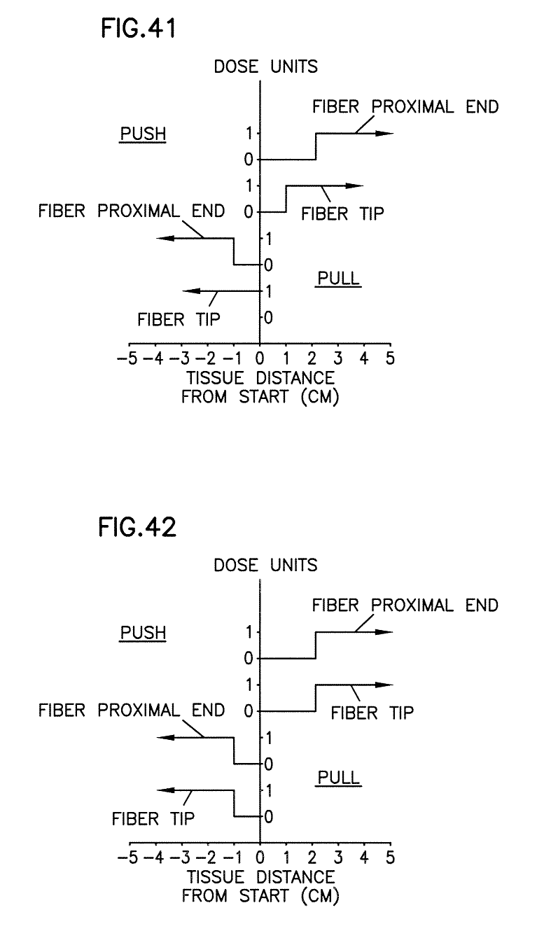

The present invention and its benefits can now be described with reference to FIGS. 41-46. Unlike the examples of FIGS. 35-40, pushes and pulls are not identical. Instead, they differ in length to create a series of staggered passes. Also, for reasons that will become apparent, they differ in the amount of laser-off time per pass.

In the embodiments of FIGS. 41-46, MAZE pattern formation preferably begins with the laser tip fully advanced to the distal end of the guide member. As a result, the first pass over tissue occurs as a “pull” pass by which the proximal end of the fiber is moved proximally by pulling on the connector 34 in FIG. 16. “Push” passes are in the opposite direction. It will be appreciated a MAZE pattern could be initiated with the laser tip fully retracted proximally in the guide member. In such a case, the first pass is a push pass.

With the present invention, one of the pass directions is longer than the other and has a longer period of laser off mode. When starting with the laser tip fully distally advanced, the longer pass is the pull pass. This is illustrated in FIGS. 41 and 42 where the pull pass is five centimeters in length and the push pass is four centimeters in length, both measured at the proximal end of the fiber at connector 34 in FIG. 16 where the push or pull force is applied. The lengths can vary from those given in the preferred embodiment.

The preferred embodiment assigns a maximum assumed hysteresis per pass. I the examples given, this is 1 cm. The start of the push and pull passes have a period of time during which laser power is off.

In a preferred embodiment, during the first two centimeters (i.e., at least equal to twice the maximum assumed hysteresis) of pull on the fiber proximal end, the laser is off. The laser is on during the last three centimeters of pull. In the push pass, during the first one centimeter (i.e., at least equal to the maximum assumed hysteresis) of push on the fiber proximal end, the laser is off. The laser is on during the last three centimeters of push. In each of FIGS. 41 and 42, the push and pull passes (and on and off states of the laser), as experienced at the proximal end of the fiber, are labeled “Fiber Proximal End” on the push and pass sides, respectively, of the graph with “0” indicating laser power off and “1” indicating laser power on.

FIG. 41 illustrates corresponding motion of the fiber tip assuming one extreme where the actual amount of hysteresis per pass equals the maximum assumed hysteresis (1 cm). The motion of the fiber tip corresponding to motion of the proximal end of the fiber is labeled “Fiber Tip”. In the pull pass, the fiber tip travels one centimeter with the fiber off. In the final three centimeters of the pull pass, the fiber is powered on. In the push pass, the entire three centimeters of fiber tip travel occur with the fiber in the power on mode.

FIG. 42 illustrates corresponding motion of the fiber tip assuming a different extreme condition. Namely, the actual amount of hysteresis per pass is zero. In both the pull and push passes, the fiber tip travels in one-to-one correspondence with movement of the proximal end of the fiber.

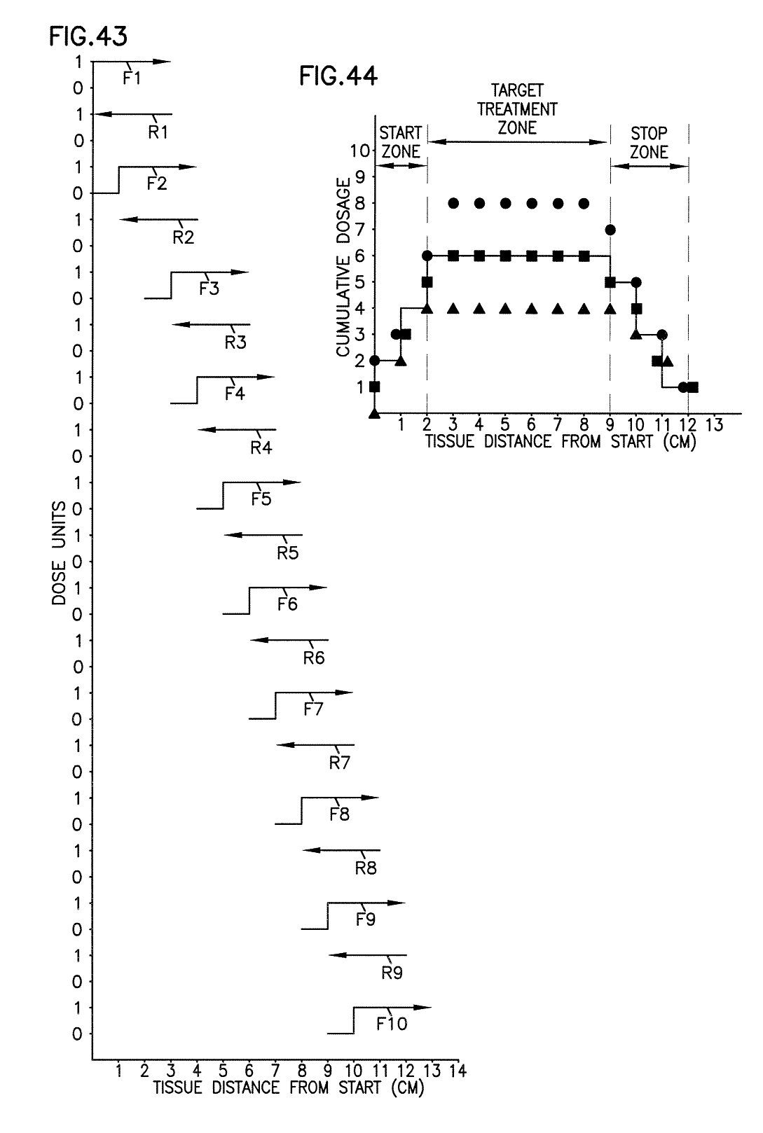

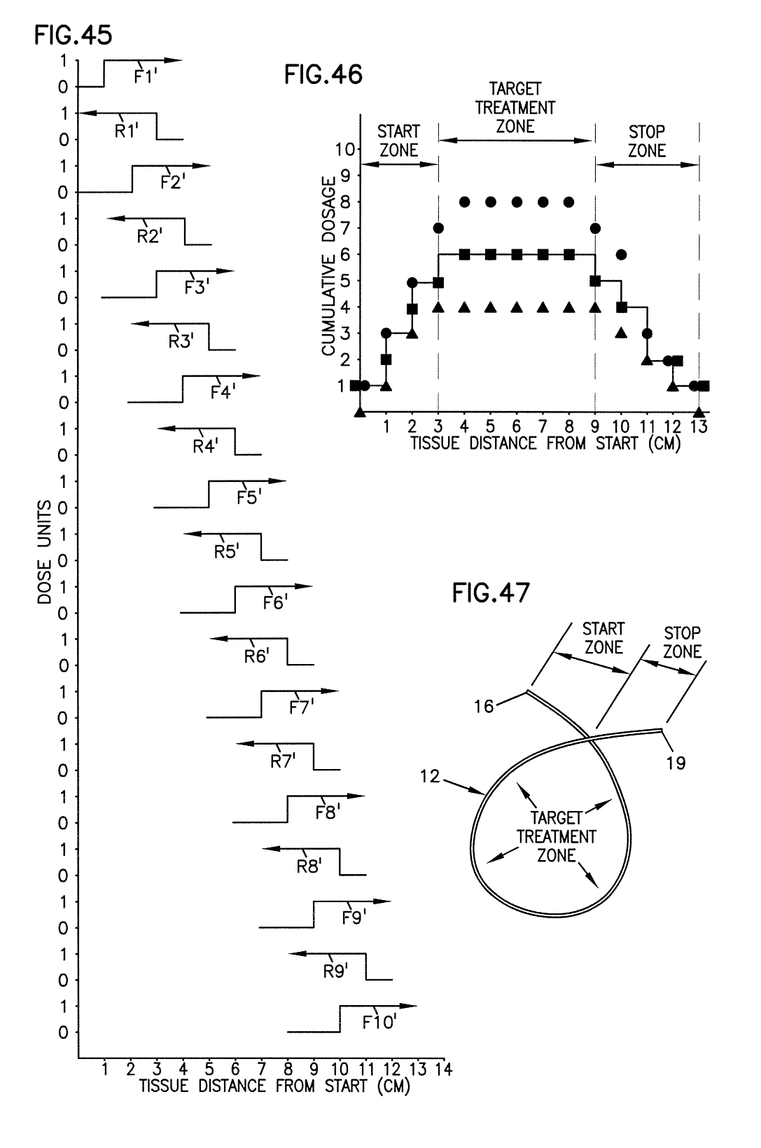

Instead of segmenting the MAZE pattern into discrete zones, the method of the present invention repeats, in staggered and overlapping sequence, pull and push passes until the fiber tip has traveled the length of the guide member. Using actual hysteresis assumptions of FIGS. 41 and 42, resulting tissue treatments are shown in FIGS. 43 and 44 (assuming the actual hysteresis per pass equals the assumed maximum hysteresis of 1 cm) and FIGS. 45 and 46 (assuming the actual hysteresis per pass equals zero).

In each of FIGS. 43 and 45, the first pull pass (F1 in FIG. 43 and F1′ in FIG. 45) differs from the remaining pull passes in that the fiber power is off only during one centimeter of proximal pull followed by three centimeters of pull with the fiber power on. As will become apparent, this provides at least some dosage to the first centimeter of tissue.

In FIG. 43, there are ten pull passes (F1-F10). At the end of each pull pass (except for the last pull pass F10), a push pass starts where the preceding pull pass ended. There are nine such pull passes (R1-R9). Except for the first pull pass F1, each pull pass starts were the preceding push pass ended.

Combined, the push and pull passes result in the fiber tip moving a total of twelve centimeters in the guide member. This is illustrative only and more pull and push passes can be provided to create a longer MAZE lesion. Further, the length of the power ON portion of each pass (three centimeters in the examples) could be shortened for more dose delivery or lengthened for less dose delivery to a tissue location. Also, dose delivery can be altered by altering the speed of travel of the fiber tip in the guide member. A faster speed results in reduced dose delivery to a tissue site.

As shown in FIG. 44, after start of the fourth pull pass F4, the tissue location beginning two centimeters from the start has received six dose units which is the desired therapy. This continues until nine centimeters from the start point after which the cumulative applied dosage steps down.

As with previously described examples, one possible variance from ideal is due to the dosage applied at the start and stop of each pass. For example, due to operating variances in equipment, tissue under the fiber tip at the start of laser power (the points X on, for example, passes F2 and F2′ in FIGS. 43 and 45) or the stop of laser power (the arrow tips in FIGS. 43 and 45) can receive one dose (numeral “1” in the vertical axis of FIGS. 43 and 45) or no dose (numeral “0” in the vertical axis of FIGS. 43 and 45).

FIG. 44 illustrates the cumulative dosage applied to tissue between tissue points at the start of the treatment and twelve centimeters from the start. While dosage applied to a 1 mm tissue segment is a desired six dose units (about 50 J in the example given) between the integer locations (i.e., locations 3, 4, 5, 6, 7 and 8 centimeters from the start of treatment) along the treatment path, dosages at these locations can vary.

The circle data points illustrate the dosage assuming tissue is treated beneath the fiber tip at the instant of power on and off (i.e., both at the tips of the arrow passes and the tails of the push passes and the start of power in the pull passes—e.g., point X in pass F2). The square data points illustrate cumulative dosage assuming tissue treatment only under the arrow tips. The triangular data points illustrate an extreme case of no treatment under either the tips of the arrows or start of power on in a pass.

After reaching the desired steady-state dosage of six dose units (i.e., after two centimeters from the start of treatment), dosage at the integer locations can vary from a high of eight dose units (about 67 J in the example given) to a low of four dose units (about 33 J in the example given). The possibility for dose variance occurs more frequently than in FIG. 38 but the possible variances are smaller in magnitude and more acceptable. Importantly, since each pull pass starts after a push pass and maximum hysteresis is accounted for, there are no possible gaps in dosing as long as actual hysteresis does not exceed the assumed maximum hysteresis. With the present invention, parameters (power level, distal tip speed, lengths of push and pull paths) can be varied to alter the steady-state dosage and the minimum and maximum variances so that the minimum dosage is adequate for forming a transmural lesion and the maximum dosage is less than a dosage presenting an unacceptable risk of tissue carbonization or perforation.

FIGS. 45 and 46 are similar to FIGS. 43 and 44 but illustrate treatment in an extreme but improbable case where the actual hysteresis is zero. Passes are similarly numbered but with the addition of an apostrophe to distinguish the examples. Dose variance data points (circles, squares and triangles) have the same meaning in FIG. 46 as in FIG. 44.

FIG. 46 illustrates the same amount of controlled and acceptable variance as in FIG. 44. High dose spikes are avoided as are gaps in treatment.

In each of FIGS. 43 and 45, the actual hysteresis experienced is the same in each pass. Of course, during a treatment, the actual hysteresis per pass can be expected to vary (but remaining less than the assumed maximum hysteresis of 1 cm). However, unlike the previous examples using a zone treatment, this is not detrimental. Each therapy pass initiates at the end of the previous therapy pass. Therefore, there are no gaps between passes.

As noted in FIGS. 44 and 46, the present method of the invention results in beginning and ending portions (referred to in the figures as “Start Zone” and “Stop Zone”) of the treated path receiving less than the desired dosage of six dose units. The method can be modified as desired to increase treatment in these zones. More easily, the guide member 12 can be positioned with ends overlapping as illustrated in FIG. 47. The ends over-lap so that the target treatment zone defines an enclosed area (e.g., surrounding pulmonary veins). With this arrangement, a path completely surrounding the enclosed area receives a full dose enhancing the likelihood of a full transmural lesion surrounding the enclosed area. The Start and Stop Zones are outside of the area and represent unnecessary lesion lengths which are of no consequence if they are less than transmural.

While the foregoing has been described in a preferred embodiment using a laser as an energy source, other devices could be employed using the method of the present invention. For example, the present invention could be used with radio-frequency, thermal, cryogenic, ultrasound or other radiant or conductive energy sources.

It has been shown how the objects of the invention have been achieved in a preferred embodiment. It is intended that such modifications and equivalents which will appear to one of ordinary skill in the art with the benefit of the teachings of the present invention shall be included within the scope of the claims.

What is claimed is:

1. An apparatus for forming a lesion in tissue along a desired ablation path, said apparatus comprising:
a movable source of energy having a distal end for applying energy to said tissue opposing said distal end and having a proximal end with an initiation of movement of said distal end delayed from an initiation of movement of said proximal end by a variable amount of discrepancy less than a maximum discrepancy,
said distal end guided for movement along said target path in response to movement of said proximal end;
a controller for operating movement of said energy source and switching said energy source between an energized mode and a non-energized mode, said controller adapted to operate said energy source as follows:
to move said proximal end in a first direction by a first distance;
during a first portion of said first distance, to operate said source in a non-energized mode;
during a second portion of said first distance, to operate said source in an energized mode;
to moving said proximal end in a second direction along said path opposite said first direction and by a distance less than said first distance;
during a first portion of said second direction, to operate said source in a non-energized mode;
during a second portion of said second direction, to operate said source in an energized mode.

2. An apparatus according to claim 1 wherein said controller is further adapted for repeating sequential ones of said first and second directions along a length of said path.

3. An apparatus according to claim 1 wherein said first portion of said first distance is not less than said maximum discrepancy.

4. An apparatus according to claim 3 wherein said first portion of said second distance is not less than said maximum discrepancy.

5. An apparatus according to claim 4 wherein said first portion of said first distance is greater than said first portion of said second distance.

6. An apparatus according to claim 5 wherein said second portions of said first and second distances are substantially equal.

7. An apparatus according to claim 6 wherein said first portion of said first distance is at least twice said first portion of said second distance.

8. An apparatus according to claim 1 wherein said energy source is an optical fiber with said proximal end connected to a laser power source.

9. An apparatus according to claim 1 wherein said energy source is a source of radiofrequency energy.

10. An apparatus according to claim 1 wherein said energy source is a source of thermal energy.

11. An apparatus according to claim 1 wherein said energy source is a cryogenic.

12. An apparatus according to claim 1 wherein said energy source is ultrasound.

13. A method for applying energy to a target path on a tissue surface using a movable source of energy having a distal end for applying energy to said tissue opposing said distal end and having a proximal end with an initiation of movement of said distal end delayed from an initiation of movement of said proximal end by a variable amount of discrepancy less than a maximum discrepancy, said distal end guided for movement along said target path in response to movement of said proximal end, said method comprising:
moving said proximal end in a first direction by a first distance;
during a first portion of said first distance, said source in a non-energized mode;
during a second portion of said first distance, said source in an energized mode;
moving said proximal end in a second direction along said path opposite said first direction and by a distance less than said first distance;
during a first portion of said second direction, said source in a non-energized mode;
during a second portion of said second direction, said source in an energized mode.

14. A method according to claim 13 further comprising repeating sequential ones of said first and second directions along a length of said path.

15. A method according to claim 14 wherein said first portion of said first distance is not less than said maximum discrepancy.

16. A method according to claim 15 wherein said first portion of said second distance is not less than said maximum discrepancy.

17. A method according to claim 16 wherein said first portion of said first distance is greater than said first portion of said second distance.

18. A method according to claim 17 wherein said second portions of said first and second distances are substantially equal.

19. A method according to claim 18 wherein said first portion of said first distance is at least twice said first portion of said second distance.

20. A method according to claim 13 wherein said energy source is an optical fiber with said proximal end connected to a laser power source.

21. A method according to claim 13 wherein said energy source is a source of radiofrequency energy.

22. A method according to claim 13 wherein said energy source is a source of thermal energy.

23. A method according to claim 13 wherein said energy source is a cryogenic.

24. A method according to claim 13 wherein said energy source is ultrasound.
